(19) United States
(12) Patent Application Publication
(10) Pub. No.: US 2020/0352521 A1
(43) Pub. Date: Nov. 12, 2020

(54) CATEGORY-BASED REVIEW AND REPORTING OF EPISODE DATA

(71) Applicant: Medtronic, Inc., Minneapolis, MN (US)

(72) Inventors: Niranjan Chakravarthy, Singapore (SG); Rodolphe Katra, Blaine, MN (US)

(21) Appl. No.: 16/832,732

(22) Filed: Mar. 27, 2020

Related U.S. Application Data
(60) Provisional application No. 62/843,762, filed on May 6, 2019.

(51) Int. Cl. A61B 5/00; A61B 5/0464; A61B 5/0205

(52) U.S. Cl. CPC A61B 5/7267; A61B 5/0205; A61B 5/7275; A61B 5/0464

(57) ABSTRACT

Techniques are disclosed for using a computing system to selectively implement different review workflows for different categories of episodes, e.g., arrhythmia episodes, stored by medical devices. The different workflows may include different combinations of one or more human and/or machine reviewers, and different decision logic for determining whether and when to present an episode to the reviewers. Machine reviewers may utilize one or more machine learning models to annotate, e.g., classify, episodes.

This application claims the benefit of U.S. Provisional Application Ser. No. 62/843,762, filed May 6, 2019, the entire content of which is incorporated herein by reference.

### FIELD

This disclosure generally relates to medical devices and, more particularly, to review and reporting of episode data collected by medical devices.

### BACKGROUND

Medical devices may be used to monitor physiological signals of a patient. For example, some medical devices are configured to sense cardiac electrogram (EGM) signals, e.g., electrocardiogram (ECG) signals, indicative of the electrical activity of the heart via electrodes. Some medical devices are configured to detect occurrences of cardiac arrhythmia, often referred to as episodes, based on the cardiac EGM and, in some cases, data from additional sensors. Example arrhythmia types include asystole, bradycardia, ventricular tachycardia, supraventricular tachycardia, wide complex tachycardia, atrial fibrillation, atrial flutter, ventricular fibrillation, atrioventricular block, premature ventricular contractions, and premature atrial contractions. The medical devices may store the cardiac EGM and other data collected during a time period including an episode as episode data. The medical device may also store episode data for a time period in response to user input, e.g., from the patient.

A computing system may obtain episode data from medical devices to allow a clinician or other user to review the episode. A clinician may diagnose a medical condition of the patient based on identified occurrences of cardiac arrhythmias within the episode. In some examples, a clinician or other reviewer may review episode data to annotate the episodes, including determining whether arrhythmias detected by the medical device actually occurred, to prioritize the episodes and generate reports for further review by the clinician that prescribed the medical device for a patient or is otherwise responsible for the care of the particular patient.

### SUMMARY

In general, review and annotation of episodes collected by medical devices facilitates the identification and reporting of episodes of interest, rather than all episodes stored by the medical device, to the clinician caring for the patient. For example, episode review may allow reporting of true bradycardia episodes stored by a medical device, and may avoid including episodes in the report that the medical device falsely stored as bradycardia due to undersensing of cardiac depolarizations. As another example, episode review and annotation may also facilitate tailored reporting of arrhythmia types or arrhythmias having characteristics of the most interest to the clinician for the particular patient.

In accordance with the techniques of the disclosure, a computing system selectively implements different review workflows for different categories of episodes stored by medical devices. The different workflows may include different combinations of one or more human and/or machine reviewers, and different decision logic for determining whether and when to present an episode to the reviewers. The different workflows may include workflows with greater reliance on human reviewers for episode categories considered more likely to be clinically significant, such as patient-triggered episodes, episodes of certain types of arrhythmias, and episodes having certain arrhythmia characteristics. Other workflows may have different degrees of automation and machine review for episodes that are less likely to be clinically significant, e.g., due to type, characteristics, or the relative prevalence of false episodes of that arrhythmia type. Machine reviewers may utilize one or more machine learning models to annotate, e.g., classify, episodes.

In this manner, the techniques of the disclosure may provide specific improvements to the field of cardiac arrhythmia classification and reporting. For example, efficiency and accuracy of review may be balanced differently for different categories of episodes, and the overall burden of episode review may be reduced.

In one example, this disclosure describes a method comprising receiving, by a computing system comprising processing circuitry and a storage medium, episode data for an episode stored by a medical device of a patient, wherein the episode data comprises a cardiac electrogram. The method further comprises categorizing, by the computing system, the episode into one category of a plurality of categories based on the episode data, the plurality of categories comprising at least a first category and a second category, and selecting, by the computing system, one review workflow from a plurality of review workflows based on the category, each of the plurality of categories associated with a respective one of the plurality of review workflows. The method further comprises selecting, by the computing system, at least one first reviewer for the episode based on the selected review workflow, providing, by the computing system, the episode data to the at least one first reviewer, and receiving, by the computing system, at least one first annotation of the episode by the at least one first reviewer, the at least one first annotation based on the provided episode data. The method further comprises determining, by the computing system, whether to provide the episode data to a second reviewer based on the selected review workflow, determining, by the computing system, whether to include the episode in an arrhythmia episode report based on the selected review workflow and the at least one first annotation, and outputting, by the computing system, the arrhythmia episode report to a user.

In another examples, a method comprises associating, by a computing system comprising processing circuitry and a storage medium, based on user input, a respective one or more episode characteristics with each of a plurality of episode categories. The plurality of categories comprises a first category associated with a manual review workflow comprising episode annotation by a human reviewer, a second category associated with an automated review workflow comprising episode annotation by a machine reviewer comprising one or more machine learning models, and a third category associated with an expedited review workflow comprising annotation by the machine reviewer and the human reviewer. The method further comprises receiving, by the computing system, episode data for an episode stored by a medical device of a patient, wherein the episode data comprises a cardiac electrogram, determining, by the computing system, at least one episode characteristic of episode based on the episode data, and categorizing, by the computing system, the episode into one of the first category, the second category, or the third category based on the at least one episode characteristic. The method further comprises providing, by the computing system, the episode data to at least one of the human reviewer or the machine reviewer based on the workflow associated with the category into which the episode was categorized, receiving, by the computing system, at least one annotation of the episode by the at least one of the human reviewer or the machine reviewer, the at least one first annotation based on the provided episode data, determining, by the computing system, whether to include the episode in an arrhythmia episode report based on the selected review workflow and the at least one annotation, and outputting, by the computing system, the arrhythmia episode report to a user.

In another example, a method comprises receiving, by a computing system comprising processing circuitry and a storage medium, episode data for a plurality of episodes stored by a plurality of medical devices of a plurality of patients, wherein the episode data for each of the episodes comprises a respective cardiac electrogram, for each of the episodes, determining, by the computing system, one or more patient characteristics or episode characteristics based on the episode data, and for each of the episodes, receiving, by the computing system, one or more annotations from a user. The method further comprises training, by the computing system, one or more machine learning models using the episode data, determined characteristics, and received annotations. The method further comprises receiving, by the computing system, episode data for at least one subsequent episode stored by at least one of the plurality of medical devices, determining, by the computing system, one or more patient characteristics or episode characteristics of the subsequent episode based on the episode data, determining, by the computing system applying the machine learning model to the determined characteristics, a review workflow for the episode, and presenting, by the computing device, the episode data to one or more reviewers for annotation based on the determined workflow.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a conceptual drawing illustrating an example of a medical device system for collecting, reviewing, and reporting episodes in accordance with the techniques of the disclosure.

FIG. 2 is a block diagram illustrating an example configuration of the implantable medical device (IMD) of FIG. 1.

FIG. 3 is a conceptual side-view diagram illustrating an example configuration of the IMD of FIGS. 1 and 2.

FIG. 4 is a functional block diagram illustrating an example configuration of the computing system of FIG. 1.

FIG. 5 is a flow diagram illustrating an example operation for episode review and reporting in accordance with the techniques of the disclosure.

FIG. 6 is a flow diagram illustrating example episode review workflows in accordance with the techniques of the disclosure.

FIG. 7 is a flow diagram illustrating example episode review workflows in accordance with the techniques of the disclosure.

FIG. 8 is a flow diagram illustrating example episode review workflows in accordance with the techniques of the disclosure.

FIG. 9 is a diagram illustrating an example multi-episode review screen that may be displayed to a reviewer during an episode review workflow in accordance with the techniques of this disclosure.

FIG. 10 is a diagram illustrating another example multi-episode review screen that may be displayed to a reviewer during an episode review workflow in accordance with the techniques of this disclosure.

FIG. 11 is a flow diagram illustrating example episode review workflows in accordance with the techniques of the disclosure.

FIG. 12 is a diagram illustrating another example multi-episode review screen that may be displayed to a reviewer during an episode review workflow in accordance with the techniques of this disclosure.

FIG. 13 is a flow diagram illustrating an example operation for configuring episode review workflows in accordance with the techniques of the disclosure.

FIG. 14 is a flow diagram illustrating an example operation for utilizing a machine learning model to configure episode review workflows in accordance with the techniques of this disclosure.

Like reference characters refer to like elements throughout the figures and description.

### DETAILED DESCRIPTION

A variety of types of implantable and external medical devices detect arrhythmia episodes based on sensed cardiac EGMs and, in some cases, other physiological parameters. External devices that may be used to non-invasively sense and monitor cardiac EGMs include wearable devices with electrodes configured to contact the skin of the patient, such as patches, watches, or necklaces. One example of a wearable physiological monitor configured to sense a cardiac EGM is the SEEQ™ Mobile Cardiac Telemetry System, available from Medtronic plc, of Dublin, Ireland. Such external devices may facilitate relatively longer-term monitoring of patients during normal daily activities, and may periodically transmit collected data, e.g., episode data for detected arrhythmia episodes, to a remote patient monitoring system, such as the Medtronic Carelink™ Network.

Implantable medical devices (IMDs) also sense and monitor cardiac EGMs, and detect arrhythmia episodes. Example IMDs that monitor cardiac EGMs include pacemakers and implantable cardioverter-defibrillators, which may be coupled to intravascular or extravascular leads, as well as pacemakers with housings configured for implantation within the heart, which may be leadless. Some IMDs that do not provide therapy, e.g., implantable patient monitors, sense cardiac EGMs. One example of such an IMD is the Reveal LINQ™ Insertable Cardiac Monitor (ICM), available from Medtronic plc, which may be inserted subcutaneously. Such IMDs may facilitate relatively longer-term monitoring of patients during normal daily activities, and may periodically transmit collected data, e.g., episode data for detected arrhythmia episodes, to a remote patient monitoring system, such as the Medtronic Carelink™ Network.

By uploading episode data from medical devices, and distributing the episode data to various users, such network services may support centralized or clinic-based arrhythmia episode review, annotation, and reporting. The episode data may include the reason the medical device recorded the episode data, e.g., whether the episode was “device triggered,” meaning the medical device recorded the episode data in response to detection of an arrhythmia, or “patient triggered,” meaning the medical device recorded the episode data in response to input from the patient or another user. The episode data may include an indication of one or more arrhythmias that the medical device detected during the episode, e.g., the detection of which triggered the medical device to record the episode data. The episode data may also include data collected by the medical device during a time period including time before and after the instant the medical device determined the one or more arrhythmias to have occurred. The episode data may include the digitized cardiac EGM during that time period, heart rates or other parameters derived from the EGM during that time period, and any other physiological parameter data collected by the medical device during the time period.

Conventionally, each episode is reviewed and annotated twice before being included in an arrhythmia report for a clinician. The first reviewer can be a junior technician who reviews the episode data to annotate the episode, e.g., identify the occurrence of one or more arrhythmias during the episode and potentially note characteristics of the arrhythmias, one-by-one in a first-in-first-out (FIFO) order. The second reviewer can be a senior/experienced ECG technician who views the first reviewer's arrhythmia annotations, makes changes if necessary, and finalizes the arrhythmia reports. Subsequent reviews can also be made if needed. In some cases, although the medical device collected the episode data in response to detecting one or more arrhythmias, the review process may determine the medical device falsely detected one or more of the arrhythmias in an episode.

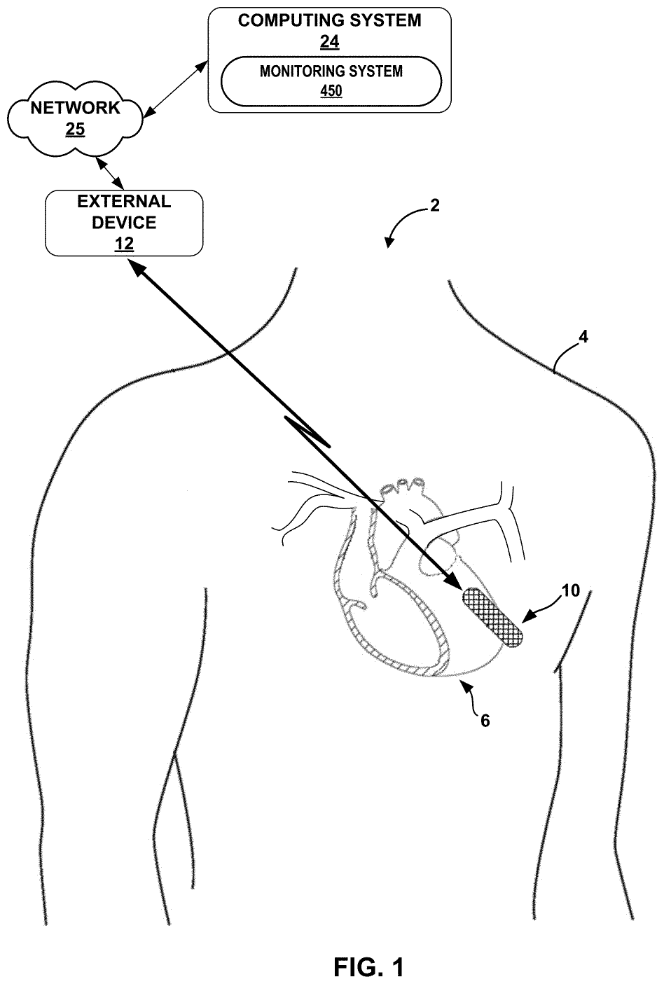

FIG. 1 is a conceptual drawing illustrating an example of a medical device system 2 for collecting, reviewing, and reporting episodes in accordance with the techniques of the disclosure. The example techniques may be used with an IMD 10, which may be in wireless communication with an external device 12. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 includes a plurality of electrodes (not shown in FIG. 1), and is configured to sense a cardiac EGM via the plurality of electrodes. In some examples, IMD 10 takes the form of the LINQ™ ICM. Although described primarily in the context of examples in which the medical device that collects episode data takes the form of an ICM, the techniques of this disclosure may be implemented in systems including any one or more implantable or external medical devices, including monitors, pacemakers, or defibrillators.

External device 12 is a computing device configured for wireless communication with IMD 10. External device 12 may be configured to communicate with computing system 24 via network 25. In some examples, external device 12 may provide a user interface and allow a user to interact with IMD 10. Computing system 24 may comprise computing devices configured to allow a user to interact with IMD 10, or data collected from IMD 10, via network 25.

External device 12 may be used to retrieve data from IMD 10 and may transmit the data to computing system 24 via network 25. The retrieved data may include values of physiological parameters measured by IMD 10, indications of episodes of arrhythmia or other maladies detected by IMD 10, episode data collected for episodes, and other physiological signals recorded by IMD 10. The episode data may include EGM segments recorded by IMD 10, e.g., due to IMD 10 determining that an episode of arrhythmia or another malady occurred during the segment, or in response to a request to record the segment from patient 4 or another user.

In some examples, computing system 24 includes one or more handheld computing devices, computer workstations, servers or other networked computing devices. In some examples, computing system 24 may include one or more devices, including processing circuitry and storage devices, that implement a monitoring system 450. Computing system 24, network 25, and monitoring system 450 may be implemented by the Medtronic Carelink™ Network or other patient monitoring system, in some examples.

Monitoring system 450 may implement the techniques of this disclosure for categorizing episodes received from medical devices, including IMD 10, selecting a review workflow based on the categorizing, and directing the episode data to reviewers according to a selected workflow. Monitoring system 450 may implement machine reviewers to automate some annotation of episode data and, based on the workflow, the selected machine reviewers may utilize one or more selected machine learning models to which the episode data is applied to, for example, to identify arrhythmias, identify characteristics of arrhythmias, and identify features of the cardiac EGM and other episode data. The machine learning models may include neural networks, deep learning models, convolutional neural networks, or other types of predictive analytics systems.

Network 25 may include one or more computing devices (not shown), such as one or more non-edge switches, routers, hubs, gateways, security devices such as firewalls, intrusion detection, and/or intrusion prevention devices, servers, computer terminals, laptops, printers, databases, wireless mobile devices such as cellular phones or personal digital assistants, wireless access points, bridges, cable modems, application accelerators, or other network devices. Network 25 may include one or more networks administered by service providers, and may thus form part of a large-scale public network infrastructure, e.g., the Internet. Network 25 may provide computing devices, such as computing system 24 and IMD 10, access to the Internet, and may provide a communication framework that allows the computing devices to communicate with one another. In some examples, network 25 may be a private network that provides a communication framework that allows computing system 24, IMD 10, and/or external device 12 to communicate with one another but isolates one or more of computing system 24, IMD 10, or external device 12 from devices external to network 25 for security purposes. In some examples, the communications between computing system 24, IMD 10, and external device 12 are encrypted.

Computing system 24 is an example of a computing system configured to receive episode data for an episode stored by a medical device of a patient, categorize the episode into one category of a plurality of categories based on the episode data, the plurality of categories comprising at least a first category and a second category, and select one review workflow from a plurality of review workflows based on the category, each of the plurality of categories associated with a respective one of the plurality of review workflows. Based on the selected workflow, computing system 24 may be configured to select at least one first reviewer for the episode, provide the episode date to the at least one first reviewer, and receive at least one first annotation of the episode by the first reviewer based on the episode data. Computing system 24 may determine whether to provide the episode data to a second reviewer based on the selected review workflow and, in some examples, based on the at least one first annotation.

Computing system 24 may categorize the episode into a first category of the plurality of categories associated with a workflow having greater human review based on the episode data indicating that the medical device stored the episode in response to user input, or in response to detecting an arrhythmia of certain types or having certain characteristics. Computing system 24 may select a more automated workflow associated with a second category for other arrhythmia types or arrhythmia characteristics, such as when true episodes of the arrhythmia type are relatively less prevalent and/or when false detections of the arrhythmia type by the medical device are relatively more prevalent than other arrhythmia types. For some workflows, computing system 24 bypasses the second reviewer based on the first annotation indicating no arrhythmia present in the episode. In some examples, the at least one first reviewer includes a first human reviewer and a first machine reviewer, and the workflow bypasses the second reviewer based on the first reviewers agreeing as to the absence of arrhythmia, agreeing as to the absence of certain monitored arrhythmias, or agreeing as to all arrhythmias identified in the episode, e.g., identifying the same one or more arrhythmias in the episode.

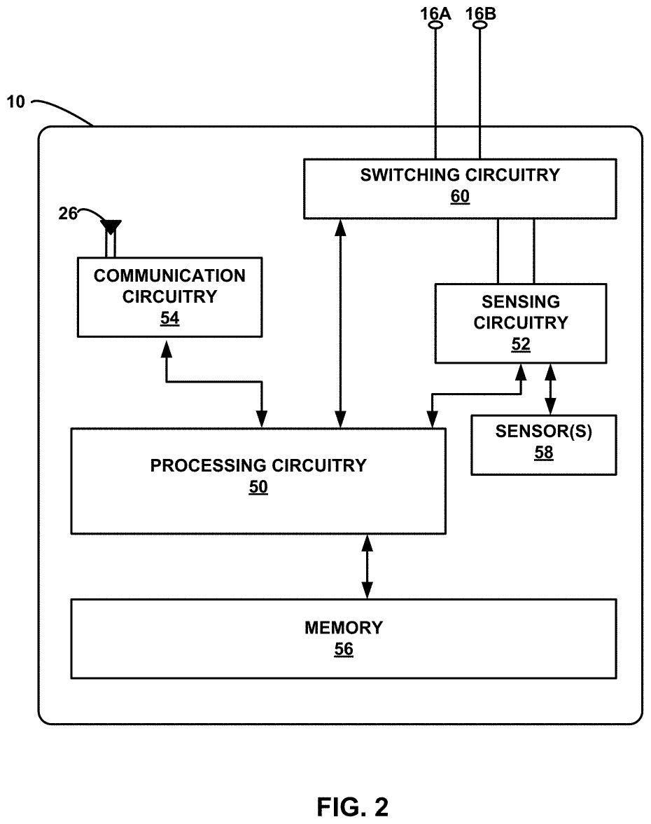

FIG. 2 is a block diagram illustrating an example configuration of IMD 10 FIG. 1. As shown in FIG. 2, IMD 10 includes processing circuitry 50, sensing circuitry 52, communication circuitry 54, memory 56, sensors 58, switching circuitry 60, and electrodes 16A, 16B (hereinafter “electrodes 16”), one or more of which may be disposed on a housing of IMD 10. In some examples, memory 56 includes computer-readable instructions that, when executed by processing circuitry 50, cause IMD 10 and processing circuitry 50 to perform various functions attributed to IMD 10 and processing circuitry 50 herein. Memory 56 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16A, 16B via switching circuitry 60 as controlled by processing circuitry 50. Sensing circuitry 52 may monitor signals from electrodes 16A, 16B in order to monitor electrical activity of a heart of patient 4 of FIG. 1 and produce cardiac EGM data for patient 4. In some examples, processing circuitry 50 may identify features of the sensed cardiac EGM to detect an episode of cardiac arrhythmia of patient 4. Processing circuitry 50 may store the digitized cardiac EGM and features of the EGM used to detect the arrhythmia episode in memory 56 as episode data for the detected arrhythmia episode. In some examples, processing circuitry 50 stores one or more segments of the cardiac EGM data, features, and other episode data in response to instructions from external device 12 (e.g., when patient 4 experiences one or more symptoms of arrhythmia and inputs a command to external device 12 instructing IMD 10 to upload the data for analysis by a monitoring center or clinician).

In some examples, processing circuitry 50 transmits, via communication circuitry 54, the episode data for patient 4 to an external device, such as external device 12 of FIG. 1. For example, IMD 10 sends digitized cardiac EGM and other episode data to network 25 for processing by monitoring system 450 of FIG. 1. The episode data may be processed by monitoring system 450 for review, annotation, and reporting of cardiac arrhythmia episodes as described in detail below.

Sensing circuitry 52 and/or processing circuitry 50 may be configured to detect cardiac depolarizations (e.g., P-waves of atrial depolarizations or R-waves of ventricular depolarizations) when the cardiac EGM amplitude crosses a sensing threshold. For cardiac depolarization detection, sensing circuitry 52 may include a rectifier, filter, amplifier, comparator, and/or analog-to-digital converter, in some examples. In some examples, sensing circuitry 52 may output an indication to processing circuitry 50 in response to sensing of a cardiac depolarization. In this manner, processing circuitry 50 may receive detected cardiac depolarization indicators corresponding to the occurrence of detected R-waves and P-waves in the respective chambers of heart. Processing circuitry 50 may use the indications of detected R-waves and P-waves for determining features of the cardiac EGM including inter-depolarization intervals, heart rate, and detecting arrhythmias, such as tachyarrhythmias and asystole. Sensing circuitry 52 may also provide one or more digitized cardiac EGM signals to processing circuitry 50 for analysis, e.g., for use in cardiac rhythm discrimination and/or to identify and delineate features of the cardiac EGM, such as QRS amplitudes and/or widths, or other morphological features.

In some examples, IMD 10 includes one or more sensors 58, such as one or more accelerometers, microphones, optical sensors, and/or pressure sensors. In some examples, sensing circuitry 52 may include one or more filters and amplifiers for filtering and amplifying signals received from one or more of electrodes 16A, 16B and/or other sensors 58. In some examples, sensing circuitry 52 and/or processing circuitry 50 may include a rectifier, filter and/or amplifier, a sense amplifier, comparator, and/or analog-to-digital converter. Processing circuitry 50 may determine values of physiological parameters of patient 4 based on signals from sensors 58, which may be used to identify arrhythmia episodes and stored as episode data in memory 56.

Communication circuitry 54 may include any suitable hardware, firmware, software or any combination thereof for communicating with another device, such as external device 12. Under the control of processing circuitry 50, communication circuitry 54 may receive downlink telemetry from, as well as send uplink telemetry to, external device 12 or another device with the aid of an internal or external antenna, e.g., antenna 26. In some examples, processing circuitry 50 may communicate with a networked computing device via an external device (e.g., external device 12) and a computer network, such as the Medtronic CareLink® Network developed by Medtronic, plc, of Dublin, Ireland.

Although described herein in the context of example IMD 10, the techniques for cardiac arrhythmia detection and episode data storage disclosed herein may be used with other types of devices. For example, the techniques may be implemented with an extra-cardiac defibrillator coupled to electrodes outside of the cardiovascular system, a transcatheter pacemaker configured for implantation within the heart, such as the Micra™ transcatheter pacing system commercially available from Medtronic PLC of Dublin Ireland, an insertable cardiac monitor, such as the Reveal LINQ™ ICM, also commercially available from Medtronic PLC, a neurostimulator, a drug delivery device, a medical device external to patient 4, a wearable device such as a wearable cardioverter defibrillator, a fitness tracker, or other wearable device, a mobile device, such as a mobile phone, a “smart” phone, a laptop, a tablet computer, a personal digital assistant (PDA), or “smart” apparel such as “smart” glasses, a “smart” patch, or a “smart” watch.

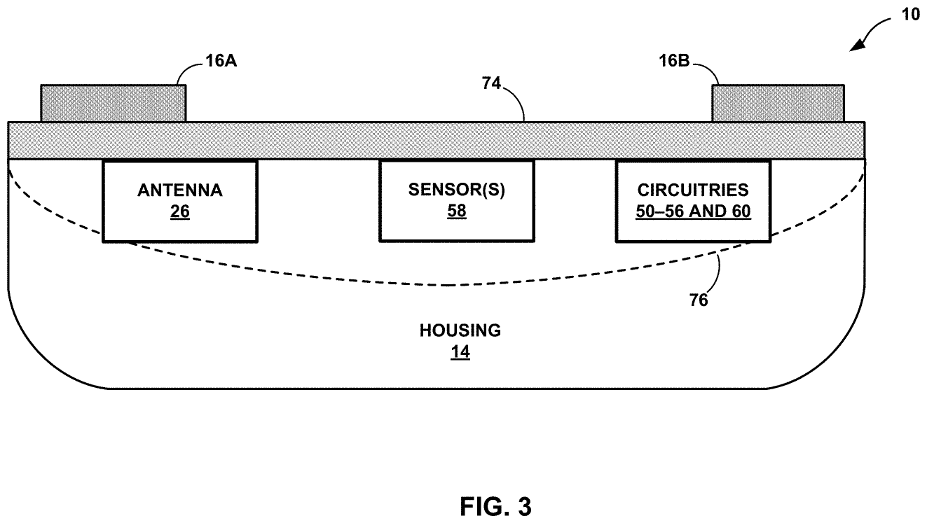

FIG. 3 is a conceptual side-view diagram illustrating an example configuration of IMD 10. In the example shown in FIG. 3, IMD 10 may include a leadless, subcutaneously-implantable monitoring device having a housing 14 and an insulative cover 74. Electrode 16A and electrode 16B may be formed or placed on an outer surface of cover 74. Circuitries 50-56 and 60, described above with respect to FIG. 2, may be formed or placed on an inner surface of cover 74, or within housing 14. In the illustrated example, antenna 26 is formed or placed on the inner surface of cover 74, but may be formed or placed on the outer surface in some examples. Sensors 58 may also be formed or placed on the inner or outer surface of cover 74 in some examples. In some examples, insulative cover 74 may be positioned over an open housing 14 such that housing 14 and cover 74 enclose antenna 26, sensors 58, and circuitries 50-56 and 60, and protect the antenna and circuitries from fluids such as body fluids.

One or more of antenna 26, sensors 58, or circuitries 50-56 may be formed on insulative cover 74, such as by using flip-chip technology. Insulative cover 74 may be flipped onto a housing 14. When flipped and placed onto housing 14, the components of IMD 10 formed on the inner side of insulative cover 74 may be positioned in a gap 76 defined by housing 14. Electrodes 16 may be electrically connected to switching circuitry 60 through one or more vias (not shown) formed through insulative cover 74. Insulative cover 74 may be formed of sapphire (i.e., corundum), glass, parylene, and/or any other suitable insulating material. Housing 14 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

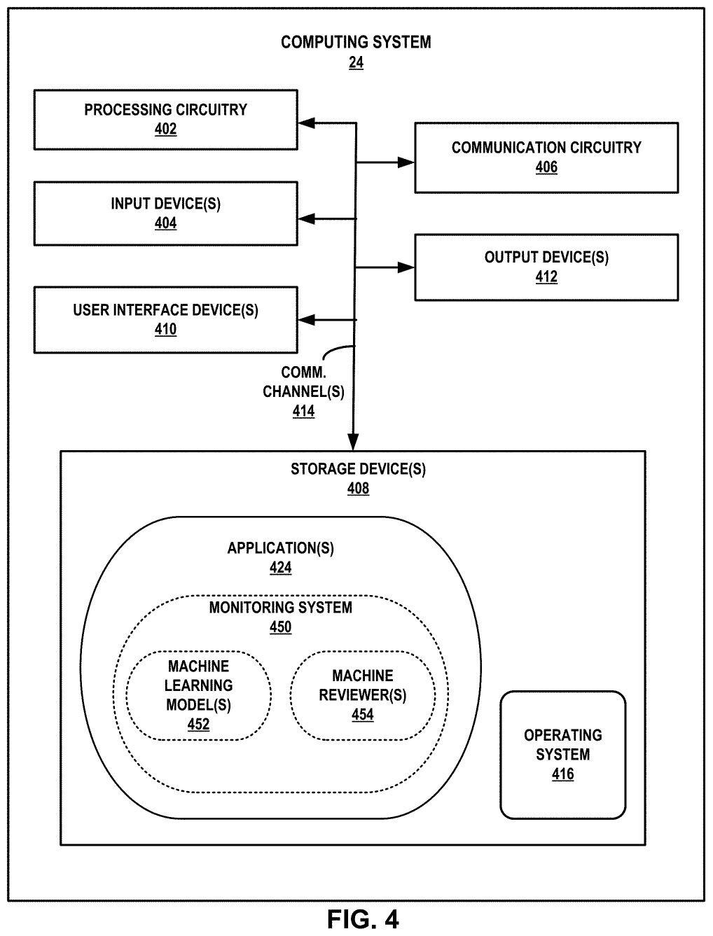

FIG. 4 is a block diagram illustrating an example configuration of computing system 24. In the illustrated example, computing system 24 includes processing circuitry 402 for executing applications 424 that include monitoring system 450 or any other applications described herein. Computing system 24 may be any component or system that includes processing circuitry or other suitable computing environment for executing software instructions and, for example, need not necessarily include one or more elements shown in FIG. 4 (e.g., input devices 404, communication circuitry 406, user interface devices 410, or output devices 412; and in some examples components such as storage device(s) 408 may not be co-located or in the same chassis as other components). In some examples, computing system 24 may be a cloud computing system distributed across a plurality of devices.

In the example of FIG. 4, computing system 24 includes processing circuitry 402, one or more input devices 404, communication circuitry 406, one or more storage devices 408, user interface (UI) device(s) 410, and one or more output devices 412. Computing system 24, in some examples, further includes one or more application(s) 424 such as monitoring system 450, and operating system 416 that are executable by computing system 24. Each of components 402, 404, 406, 408, 410, and 412 are coupled (physically, communicatively, and/or operatively) for inter-component communications. In some examples, communication channels 414 may include a system bus, a network connection, an inter-process communication data structure, or any other method for communicating data. As one example, components 402, 404, 406, 408, 410, and 412 may be coupled by one or more communication channels 414.

Processing circuitry 402, in one example, is configured to implement functionality and/or process instructions for execution within computing system 24. For example, processing circuitry 402 may be capable of processing instructions stored in storage device 408. Examples of processing circuitry 402 may include, any one or more of a microprocessor, a controller, a digital signal processor (DSP), an application specific integrated circuit (ASIC), a field-programmable gate array (FPGA), or equivalent discrete or integrated logic circuitry.

One or more storage devices 408 may be configured to store information within computing device 400 during operation. Storage device 408, in some examples, is described as a computer-readable storage medium. In some examples, storage device 408 is a temporary memory, meaning that a primary purpose of storage device 408 is not long-term storage. Storage device 408, in some examples, is described as a volatile memory, meaning that storage device 408 does not maintain stored contents when the computer is turned off. Examples of volatile memories include random access memories (RAM), dynamic random access memories (DRAM), static random access memories (SRAM), and other forms of volatile memories known in the art. In some examples, storage device 408 is used to store program instructions for execution by processing circuitry 402. Storage device 408, in one example, is used by software or applications 424 running on computing system 24 to temporarily store information during program execution.

Storage devices 408, in some examples, also include one or more computer-readable storage media. Storage devices 408 may be configured to store larger amounts of information than volatile memory. Storage devices 408 may further be configured for long-term storage of information. In some examples, storage devices 408 include non-volatile storage elements. Examples of such non-volatile storage elements include magnetic hard discs, optical discs, floppy discs, flash memories, or forms of electrically programmable memories (EPROM) or electrically erasable and programmable memories (EEPROM).

Computing system 24, in some examples, also includes communication circuitry 406 to communicate with other devices and systems, such as IMD 10 and external device 12 of FIG. 1, as well as other networked client computing devices of various users. Communication circuitry 406 may include a network interface card, such as an Ethernet card, an optical transceiver, a radio frequency transceiver, or any other type of device that can send and receive information. Other examples of such network interfaces may include 3G and WiFi radios.

Computing system 24, in one example, also includes one or more user interface devices 410. User interface devices 410, in some examples, are configured to receive input from a user through tactile, audio, or video feedback. Examples of user interface devices(s) 410 include a presence-sensitive display, a mouse, a keyboard, a voice responsive system, video camera, microphone or any other type of device for detecting a command from a user. In some examples, a presence-sensitive display includes a touch-sensitive screen.

One or more output devices 412 may also be included in computing system 24. Output device 412, in some examples, is configured to provide output to a user using tactile, audio, or video stimuli. Output device 412, in one example, includes a presence-sensitive display, a sound card, a video graphics adapter card, or any other type of device for converting a signal into an appropriate form understandable to humans or machines. Additional examples of output device 412 include a speaker, a cathode ray tube (CRT) monitor, a liquid crystal display (LCD), or any other type of device that can generate intelligible output to a user.

Computing system 24 may include operating system 416. Operating system 416, in some examples, controls the operation of components of computing system 24. For example, operating system 416, in one example, facilitates the communication of one or more applications 424 and monitoring system 450 with processing circuitry 402, communication circuitry 406, storage device 408, input devices 404, user interface devices 410, and output devices 412.

Applications 424 may also include program instructions and/or data that are executable by computing device 400. Example application(s) 424 executable by computing device 400 may include monitoring system 450. Other additional applications not shown may alternatively or additionally be included to provide other functionality described herein and are not depicted for the sake of simplicity.

In accordance with the techniques of the disclosure, computing system 24 receives episode data for episodes stored by medical devices, such as IMD 10, via communication circuitry 406. Processing circuitry 402 may store the episode data for the episodes in storage device 408. The episode data may have been collected by the medical devices in response to the medical devices detecting arrhythmias and/or user input directing the storage of episode data.

Monitoring system 450, as implemented by computing system 24 including processing circuitry 402 and storage device 408, controls the review and annotation of the episodes, and generation of reports of the episodes subsequent to the annotation for clinician review. Monitoring system 450 may utilize input devices 404, output devices 412, and/or communication circuitry 406 to direct episode data to one or more human reviewers, e.g., via one or more graphical user interfaces presented on output devices 412 or other client computing devices, and to receive annotations of the episode data from the one or more human reviewers. Depending on the review workflow selected for an episode, monitoring system 450 may also implement one or more machine reviewers 454 to review and annotate the episode data.

Based on which review workflow is selected, machine reviewers 454 may select one or more machine learning models 452 and apply the episode data as inputs to the one or more selected machine learning models 452. Machine learning models 452 may be configured to output values indicative of the probability that the episode data includes arrhythmia or not, or the probabilities that the episode data includes arrhythmias of certain types that the machine learning models are configured to detect, as examples. Machine reviewers 454 may apply configurable thresholds to the probability values to annotate the episode as including one or more arrhythmia types, e.g., by providing an indication of an arrhythmia type or other classification if the probability of the classification exceeds a threshold. Monitoring system 450 may utilize different machine learning models 452 for different review workflows and/or based on which arrhythmias or characteristics of arrhythmias are of interest to a particular clinician and/or for a particular patient.

Machine learning models 452 may also be configured to identify features within cardiac EGM data of the episode, such as depolarizations, and machine reviewers 454 may classify or otherwise annotate the episode data based on the features. In some examples, machine reviewers 454 may be able to identify a time during an episode that one or more arrhythmias likely occurred based on the output of an intermediate layer of a machine learning model 452 used to classify the time series of episode data. Machine learning models 452 may be trained on training episode data sets having known classifications as determined by human reviewers. Machine learning models 452 may include, as examples, neural networks, such as deep neural networks, which may include convolutional neural networks, multi-layer perceptrons, and/or echo state networks, as examples.

Monitoring system 450 categorizes each of the episodes into one category of a plurality of categories based on the episode data, and selects one review workflow from a plurality of review workflows based on the category. Based on the selected workflow, monitoring system 450 may be configured to select at least one first reviewer for the episode, provide the episode date to the at least one first reviewer, and receive at least one first annotation of the episode by the first reviewer based on the episode data. Monitoring system 450 may determine whether to provide the episode data to a second reviewer based on the selected review workflow and, in some examples, based on the at least one first annotation. In some examples, the at least one first reviewer includes a first human reviewer and a first machine reviewer 454, which may utilize a selected one or more machine learning models 452 to determine the annotations of the episode data.

Based on reporting criteria and the annotations by the first and, in some cases, second reviewers according to the selected workflow, monitoring system 450 may determine whether to include the episode data for episodes in an arrhythmia report. Monitoring system 450 may generate the arrhythmia report for review by a clinician caring for a particular one or more patients and may select episode data from medical devices of the one or more patients to include in the report based on reporting criteria. Example reporting criteria include whether the episode was annotated as including any arrhythmias, or as including arrhythmias of certain types or having certain characteristics. The arrhythmia types and characteristics of interest may be configured by the clinician or other user. Examples of arrhythmia types include asystole, bradycardia, ventricular tachycardia, supraventricular tachycardia, wide complex tachycardia, atrial fibrillation, atrial flutter, ventricular fibrillation, atrioventricular block, premature ventricular contractions, and premature atrial contractions. Examples of arrhythmia characteristics include heart depolarization rates satisfying a threshold or certain morphological characteristics of the cardiac EGM being present.

In some examples, the criteria for categorizing episodes, the configuration of workflows, and the reporting criteria may be user-configurable to present episodes of interest, e.g., according to the monitoring requirements or monitoring reasons specified for the clinician, clinic, or patient, with a high degree of confidence of correct classification, and to provide efficient review of other episode types. In some examples, the reporting criteria may customizable, e.g., patient specific. For example, a physician might request reporting of bradycardia episodes <=30 beats-per-minute in certain patients and <=40 beats-per-minute in other patients. Monitoring system 450 may compare the annotations made according to the selected workflow to such reporting criteria when determining whether to include an episode in a report to the physician.

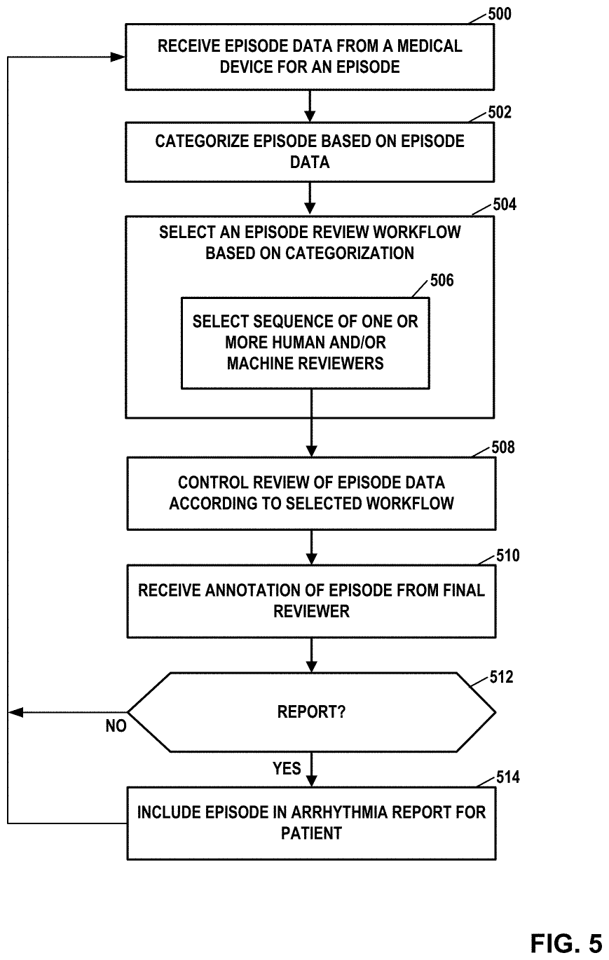

FIG. 5 is a flowchart illustrating an example operation for episode review and reporting in accordance with the techniques of the disclosure. For convenience, FIGS. 5-14 are described with respect to medical device system 2 described with respect to FIGS. 1-4, and as being performed by monitoring system 450. The example techniques illustrated by FIGS. 5-14 may be performed by any computing device comprising processing circuitry and one or more storage devices.

According to the example operation of FIG. 5, monitoring system 450 receives episode data from IMD 10 for an episode (500). Monitoring system 450 categorizes the episode based on the episode data (502). In various examples, monitoring system 450 categorizes the episodes based on whether the episode data was recorded by IMD 10 in response to detecting an arrhythmia or user input, which of a plurality of types the arrhythmia detected by IMD 10 was, including the relative prevalence of the type or of false detections of that type, or whether the arrhythmia detected by IMD 10 exhibited certain characteristics of interest.

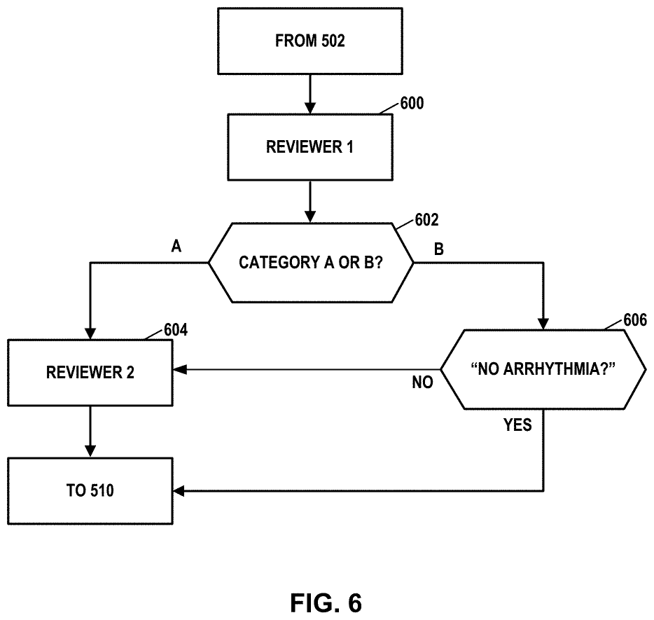

Monitoring system 450 further selects an episode review workflow for the episode based on the categorization (504). In some examples, each episode category may be associated with a respective review workflow. As part of the selection of the workflow, monitoring system 450 may select a sequence of one or more human and/or machine reviewers to review and annotate the episode data (506). Different review workflows may specify different combinations of one or more human and/or machine reviewers, and different decision logic for determining whether and when to present an episode to the reviewers. Although the example operations of FIG. 6-12 include two categories of episodes and selection between two possible workflows, examples consistent with this disclosure may include any number of categories and associated workflows. Additionally, although the examples include first and second reviewers, examples consistent with this disclosure may include any number of reviewers.

Monitoring system 450 controls the review of episode data by the selected reviewers according to the selected workflow (508). Monitoring system 450 also receives annotations of the episode data by the selected reviewers, including a final annotation by a final reviewer, based on the selected review workflow (510). For example, based on the selected workflow, monitoring system 450 may control the timing and format of presentation of episode data to various human reviewers via output devices 412 and receipt of annotations from the human reviewers via input devices 404, as well as executing selected machine reviewers 454 and machine learning models 452 to determine machine annotations of the episode data.

Based on the final annotation and the selected workflow, monitoring system 450 determines whether the episode meets reporting criteria, which may be tailored according to the requirements of a monitoring center, clinic, or clinician, or for a particular patient, to be included in an arrhythmia report (512). If the episode qualifies for inclusion in a report (YES of 512), monitoring system 450 includes the episode data and annotation in the report (514). In either case, monitoring system 450 may continue with receipt of episode data for another episode from IMD 10 (500).

FIG. 6 is a flow diagram illustrating example episode review workflows in accordance with the techniques of the disclosure. The operation of FIG. 6 may correspond to one example implementation of actions 504-508 of the example operation of FIG. 5.

According to the example of FIG. 6, monitoring system 450 provides the episode data to a first reviewer, e.g., a first human reviewer, and receives the first annotation from the first reviewer (600). In the example of FIG. 6, the first reviewer is denoted as “REVIEWER 1.” Monitoring system 450 further determines whether the episode was categorized as a first category or second category, category A or category B, based on the episode data (602). In some examples, category A includes all episodes stored by IMD 10 in response to a patient trigger, and category B includes all episodes stored by IMD 10 in response to detecting an arrhythmia. In other examples, category A includes all episodes stored by IMD 10 in response to a patient trigger and episodes stored by IMD 10 in response to detecting arrhythmias of one or more types or having one or more characteristics. In such example, category B may include episodes stored by IMD 10 in response to detecting arrhythmias of other types or not having the certain characteristics.

For episodes in category A, monitoring system 450 provides the episode data and the first annotation to a second reviewer (604), who either agrees with the first annotation or provides a revised annotation. In the example of FIG. 6, the second reviewer is denoted as “REVIEWER 2.” For episodes in category B, monitoring system 450 determines whether the first annotation indicates no arrhythmia (606). Based on the first annotation indicating no arrhythmia present in the episode (YES of 606), monitoring system 450 may bypass the second reviewer. Based on the first annotation indicating an arrhythmia is present in the episode (NO of 606), monitoring system 450 may present the episode data and the first annotation to a second reviewer (604), which either agrees the first annotation or provides a revised annotation.

In some examples, patient-triggered episodes are in a category that receives two stages of human review since the presence or absence of arrhythmias associated with patient-triggered episodes may have relatively high diagnostic relevance for appropriate therapy. In some examples, if the first reviewer considers that there are no arrhythmias present in an episode, or that none of the arrhythmia types or arrhythmia characteristics of interest for the selected review workflow are present, the episode may be finalized without subsequent review since the absence of arrhythmias does not need multiple reviews or confirmations. The first reviewer can choose to have a small set of episodes reviewed again if they are unsure about arrhythmia absence.

An annotation or classification of an episode as “no arrhythmia” may mean that no arrhythmias occurred during the episode, or that none of the arrhythmia types or arrhythmia characteristics of interest, and which the reviewers are instructed to identify according to the selected review workflow, are present. Machine reviewers 454 and machine learning models 452 selected for a particular review workflow, for example, may be configured to identify a certain set of arrhythmia types and/or arrhythmias having certain characteristics. Similarly, human reviewers may be instructed to look for certain arrhythmia types and/or arrhythmias having certain characteristics, and not annotate episodes based on other arrhythmias. Arrhythmia types or characteristics of interest may be determined based on the requirements or preferences of a particular monitoring center, clinic, or clinician, or for a particular patient or class of patients. While the definition of “no arrhythmia” can change per such requirements or preferences, one example is the absence of atrial fibrillation or atrial flutter, wide complex tachycardias, AV blocks, bradycardia, asystole, and tachycardia.

In some examples, only certain auto-triggered episodes are reviewed by the first reviewer to determine whether the episode may be annotated as “no arrhythmia,” while others go through the two-reviewer workflow. The auto-triggered episodes reviewed by the first reviewer for absence of arrhythmias can be auto-triggered episodes with an arrhythmia type which has a low arrhythmia prevalence (based on historical estimates), e.g., bradycardia auto-triggers from a patient who is exhibiting QRS undersensing. The expected efficiency savings of having certain episodes possibly be reviewed by a single reviewer is a function of the prevalence of different types of episodes. For example, if 5% of all episodes are patient-triggered and 95% of episodes are auto-triggered, and 50% of auto-triggers may be classified “no arrhythmia,” then 47.5% of all episodes get reviewed once. In this example, a 23% review efficiency improvement may be achieved without reducing diagnostic yield.

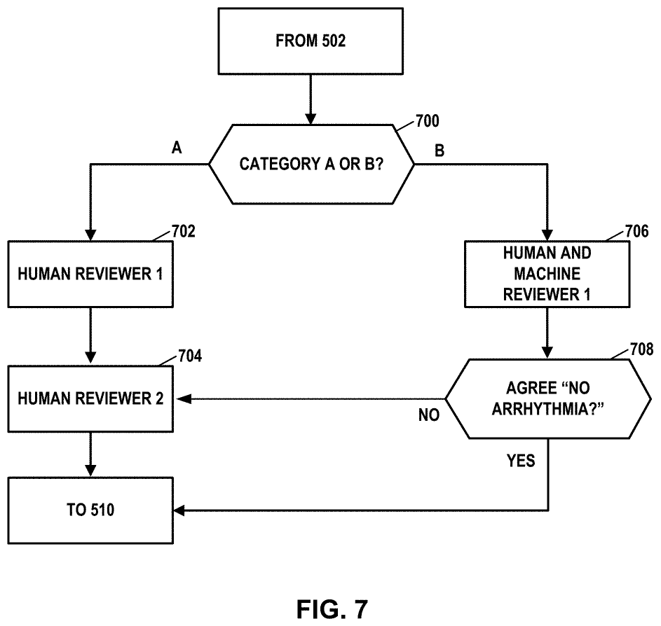

FIG. 7 is a flow diagram illustrating example episode review workflows in accordance with the techniques of the disclosure. The operation of FIG. 7 may correspond to one example implementation of actions 504-508 of the example operation of FIG. 5.

According to the example of FIG. 7, monitoring system 450 determines whether the episode was categorized as a first category or second category, category A or category B, based on the episode data (700). In some examples, category A includes all episodes stored by IMD 10 in response to a patient trigger, and category B includes all episodes stored by IMD 10 in response to detecting an arrhythmia. In other examples, category A includes all episodes stored by IMD 10 in response to a patient trigger and episodes stored by IMD 10 in response to detecting arrhythmias of one or more types or having one or more characteristics. In such examples, category B may include episodes stored by IMD 10 in response to detecting arrhythmias of other types or not having the certain characteristics.

For episodes in category A, monitoring system 450 provides the episode data to a first human reviewer (702), who provides monitoring system 450 a first annotation of the episode data. In the example of FIG. 7, the first human reviewer is denoted as “HUMAN REVIEWER 1.” Monitoring system 450 provides the episode data and first annotation to a second reviewer (704), which either agrees the first annotation or provides a revised annotation. In the example of FIG. 7, the second reviewer is denoted as “HUMAN REVIEWER 2.”

For episodes in category B, monitoring system 450 provides the episode data to a first human reviewer, which may be the same as or different from the first human reviewer that receives episode data for episodes in category A, and to a first machine reviewer 454 (706). The first machine reviewer 454 may apply one or more machine learning models 452 to at least some of the episode data and, based on the output of the one or more machine learning models 452, determine a first annotation that indicates whether or not the episode is annotated or classified “no arrhythmia.” Monitoring system 450 may provide the episode data to the first human reviewer and the first machine reviewer 454 in parallel, or may first provide the episode data to the first machine reviewer 454 and may allow the first human reviewer to review the annotation made by the first machine reviewer 454. In some examples, monitoring system 450 may first provide the episode date to the first human reviewer, and provide the annotation from the first human reviewer as input to the first machine reviewer 454 for its analysis of the episode.

In any of these cases, monitoring system 450 determines whether the first annotations both indicate “no arrhythmia” (708). Based on the first annotations agreeing on the annotation “no arrhythmia” for the episode (YES of 708), monitoring system 450 may bypass the second reviewer. Based on at least one of the first annotations not indicating “no arrhythmia” for the episode (NO of 708), monitoring system 450 may present the episode data and the first annotation to a second human reviewer (704), which either agrees with the first annotations or provides a revised annotation.

In some examples, auto-triggered episodes are processed in parallel by a machine reviewer 454 with one or more machine learning models 452 and a first human reviewer. If monitoring system 450 determines that both the first human reviewer and the machine reviewer 454 have annotated or flagged an episode as “no arrhythmia,” then monitoring system 450 may finalize the episode without subsequent review, e.g., by a second human reviewer. Parallel processing by a machine reviewer 454 can be considered to be an independent evaluation of the arrhythmia content of the episode data, particularly in examples in which the first human reviewer is blinded to the annotation by the machine reviewer 454.

A user or monitoring system 450 may configure the criteria for inclusion in category B to include arrhythmia types where the prevalence of “no arrhythmia” annotation, e.g., due to false detection by IMD 10, is not low. Monitoring system 450 may select the machine learning model 452 and configure machine reviewer 454 to have a high specificity such that the net positive-predictive value (PPV) of “no arrhythmia” detection is higher than the prevalence of no arrhythmia episodes for arrhythmias of the type(s) included in category B. In some examples, monitoring system 450 determines a prevalence of false detection of arrhythmias of the arrhythmia type by IMD 10, determines a positive predictive value of no arrhythmia for machine learning models 452, comparing the prevalence of false detection to the positive predictive values, and selecting the machine learning model 452 for use by machine reviewer 454 for episodes of category B based on the comparison.

For example, if the prevalence of “no arrhythmia” in all auto-triggers is 10%, and the prevalence of “no arrhythmia” for atrial fibrillation episodes detected by IMD 10 is 40%, atrial fibrillation auto-trigger episodes can be chosen for category B and its associated review workflow. Furthermore, assuming three possible machine learning models 452, of which the first has a specificity of 99% and sensitivity of 50% in detecting “no arrhythmia,” the second has a specificity of 80% and sensitivity of 80% in detecting “no arrhythmia,” and the third has a specificity and sensitivity of 50% in detecting “no arrhythmia,” monitoring system 450 may choose the first machine learning model 452 since the overall PPV is 97% which is higher than that of the second model (72%) and the third model (40%).

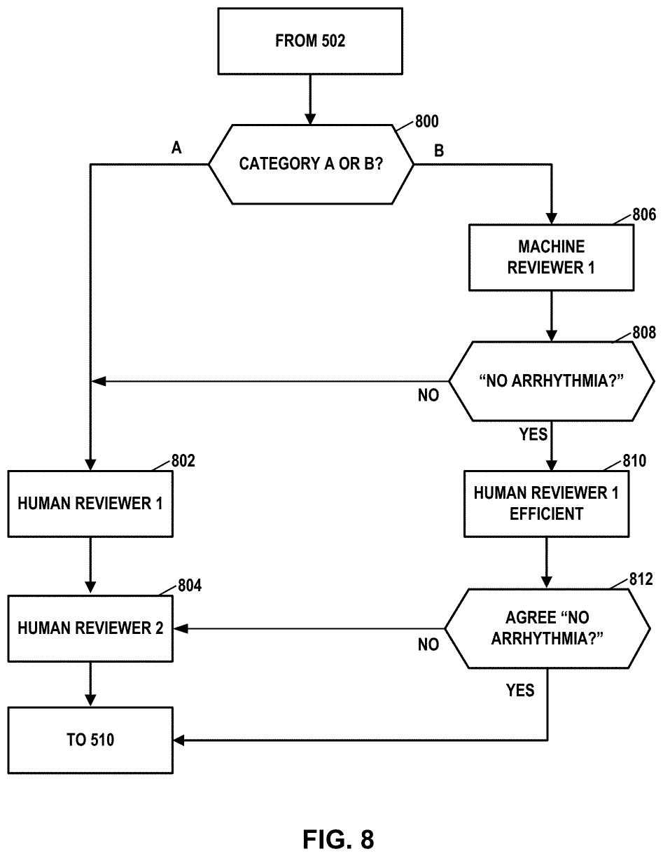

FIG. 8 is a flow diagram illustrating example episode review workflows in accordance with the techniques of the disclosure. The operation of FIG. 8 may correspond to one example implementation of actions 504-508 of the example operation of FIG. 5.

According to the example of FIG. 8, monitoring system 450 determines whether the episode was categorized as a first category or second category, category A or category B, based on the episode data (800). In some examples, category A includes all episodes stored by IMD 10 in response to a patient trigger, and category B includes all episodes stored by IMD 10 in response to detecting an arrhythmia. In other examples, category A includes all episodes stored by IMD 10 in response to a patient trigger and episodes stored by IMD 10 in response to detecting arrhythmias of one or more types or having one or more characteristics. In such examples, category B may include episodes stored by IMD 10 in response to detecting arrhythmias of other types or not having the certain characteristics.

For episodes in category A, monitoring system 450 provides the episode data to a first human reviewer (802), which provides monitoring system 450 a first annotation of the episode data. In the example of FIG. 8, the first human reviewer is denoted as “HUMAN REVIEWER 1.” Monitoring system 450 provides the episode data and first annotation to a second human reviewer (804), who either agrees with the first annotation or provides a revised annotation. In the example of FIG. 8, the second human reviewer is denoted as “HUMAN REVIEWER 2.”

For episodes in category B, monitoring system 450 provides the episode data to a first machine reviewer 454 (806). In the example of FIG. 8, the first machine reviewer 454 is denoted as “MACHINE REVIEWER 1.” The first machine reviewer 454 may apply one or more machine learning models 452 to at least some of the episode data and, based on the output of the one or more machine learning models 452, determine a first annotation that indicates whether or not the episode is classified “no arrhythmia” (808). Based on the first machine reviewer 454 determining that at least one arrhythmia, or arrhythmia of interest, is present in the episode data (NO of 808), monitoring system 450 may provide the episode data and the annotation by machine reviewer 454 to a first human reviewer (802), which provides monitoring system 450 a first human annotation of the episode data. Monitoring system 450 provides the episode data and annotations to a second human reviewer (804), which either agrees the first annotations or provides a revised annotation.

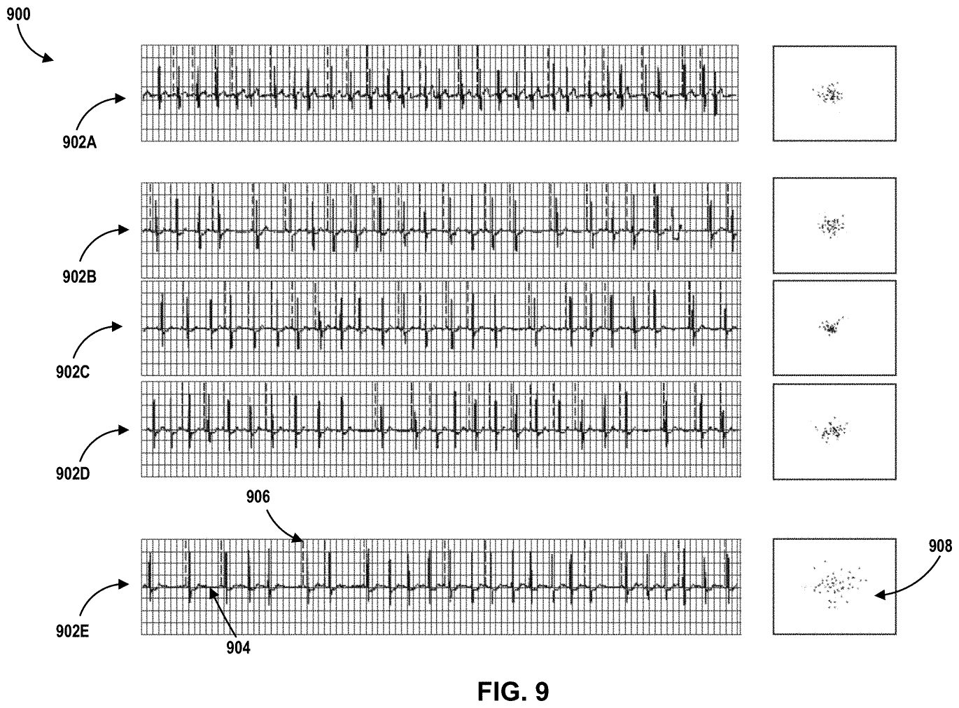

Based on the first machine reviewer 454 annotating the episode “no arrhythmia” (YES of 808), monitoring system 450 may provide the episode data and the annotation from the first machine reviewer 454 to a first human reviewer, which may be the same as or different then the first human reviewer that receives episode data for episodes in category A, for efficient review (810). For the efficient review, monitoring system 450 may present episode data for multiple episodes having commonality, e.g., same arrhythmia type or characteristics, to the first human reviewer via a multi-episode review screen, examples of which are described with respect to FIGS. 9, 10, and 12. Monitoring system 450 receives an annotation of the episode data from the first human reviewer's efficient review, and determines whether there is agreement with the machine reviewer that the episode data include no arrhythmia (812). Based on the first human reviewer annotation indicating agreement that there is “no arrhythmia” (YES of 812), monitoring system 450 bypasses the second human reviewer. Based on the first human reviewer annotation identifying one or more arrhythmias, or arrhythmias of interest, in the episode data, or being unsure regarding the presence or absence of such arrhythmias (NO of 812), monitoring system 450 provides the annotations and episode data to a second human reviewer (804).

In some examples, monitoring system 450 uses a machine reviewer 454 to analyze auto-triggered episodes with one or more machine learning models 452. Episodes where machine reviewer 454 detects any arrhythmias may go through first and second human reviewers, e.g., with the first human reviewer is blinded to the machine reviewer annotation. Episodes where machine reviewer 454 detects no arrhythmias may be presented to the first reviewer in a multi-episode review screen for expedited review. Monitoring system 450 may finalize episodes where the first human reviewer agrees on no arrhythmias, e.g., exclude the episode from an arrhythmia report, and forward episodes where the first reviewers disagree to a second human reviewer.

FIG. 9 is a diagram illustrating an example multi-episode review screen 900 that may be displayed to a reviewer during an episode review workflow in accordance with the techniques of this disclosure. In the illustrated example, review screen 900 includes episode data for episodes 902A-902E (collectively “episodes 902”), including the cardiac EGMs 904 for these episodes. Screen 900 may include episodes 902 from different patients, and episodes 902B-902D may be grouped together due to their being from the same patient, for example. In the example of FIG. 9, episodes 902 are auto-triggered episodes that medical devices stored based on detecting atrial fibrillation, and that a machine reviewer 454 annotated as “no arrhythmia,” e.g., due to not including atrial fibrillation or any other arrhythmia of interest.

In addition to the episode data, screen 900 may include other information, such as annotations, e.g., by machine reviewer 454, that may aid in efficient review by the first human reviewer. The annotations may include features of the episode data identified by, and/or information derived from the episode data by machine reviewer 454, e.g., by one or more machine learning models 452. The information may be specific to the arrhythmia type that the medical devices detected, and which machine reviewer 454 determined was falsely detected, e.g., atrial fibrillation in the illustrated example. In the illustrated example, monitoring system 450 includes P-wave markers 906 and R-R variability scatter plots 908 determined by machine reviewer 454 based on the episode data. Other atrial fibrillation specific information that could be included in an efficient episode review screen includes information indicating variance of depolarization morphology. Generally, P-waves being present in conjunction with R-waves, low R-R variance, or high morphology variance indicate absence of atrial fibrillation.

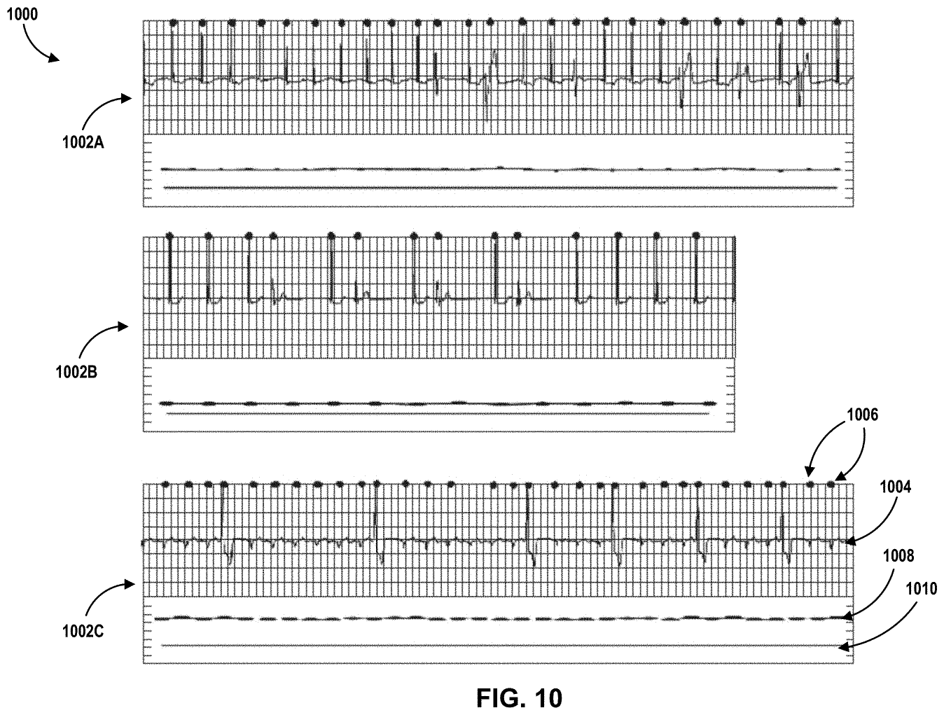

FIG. 10 is a diagram illustrating another example multi-episode review screen 1000 that may be displayed to a reviewer during an episode review workflow in accordance with the techniques of this disclosure. In the illustrated example, review screen 1000 includes episode data for episodes 1002A-1002C (collectively “episodes 1002”), including the cardiac EGMs 1004 for these episodes. In the example of FIG. 10, episodes 1002 are auto-triggered episodes that medical devices stored based on detecting bradycardia, and that a machine reviewer 454 annotated as “no arrhythmia,” e.g., due to not including bradycardia or any other arrhythmia of interest.

In addition to the episode data, screen 1000 may include other information, such as annotations, e.g., by machine reviewer 454, that may aid in efficient review by the first human reviewer. The annotations may include features of the episode data identified by, and/or information derived from the episode data by machine reviewer 454, e.g., by one or more machine learning models 452. The information may be specific to the arrhythmia type that the medical devices detected, and which machine reviewer 454 determined was falsely detected, e.g., bradycardia in the illustrated example. In the illustrated example, monitoring system 450 includes R-wave markers 1006 for R-waves detected by machine reviewer 454, a plot of R-R intervals or heart rate 1008 on a beat-to-beat basis determined based on R-wave markers 1006, and an R-R interval or rate threshold 1010 on screen 1000 for each episode 1002. Threshold 1000 may be patient specific. The information presented on screen 1000 illustrates to a human reviewer performing efficient review why the machine reviewer 454 determined that the detection of bradyarrhythmia by the medical device was mistaken.

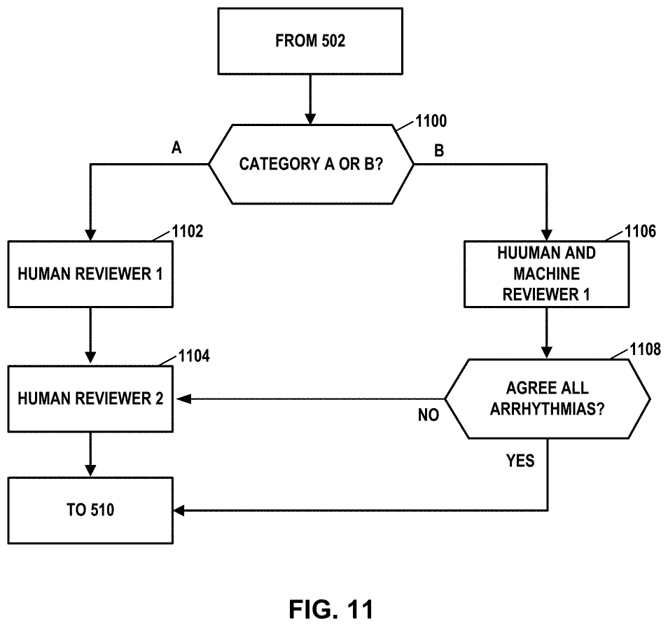

FIG. 11 is a flow diagram illustrating example episode review workflows in accordance with the techniques of the disclosure. The operation of FIG. 11 may correspond to one example implementation of actions 504-508 of the example operation of FIG. 5.

According to the example of FIG. 11, monitoring system 450 determines whether the episode was categorized as a first category or second category, category A or category B, based on the episode data (1100). In some examples, category A includes all episodes stored by IMD 10 in response to a patient trigger, and category B includes all episodes stored by IMD 10 in response to detecting an arrhythmia. In other examples, category A includes all episodes stored by IMD 10 in response to a patient trigger and episodes stored by IMD 10 in response to detecting arrhythmias of one or more types or having one or more characteristics. In such examples, category B may include episodes stored by IMD 10 in response to detecting arrhythmias of other types or not having the certain characteristics.

For episodes in category A, monitoring system 450 provides the episode data to a first human reviewer (1102), which provides monitoring system 450 a first annotation of the episode data. Monitoring system 450 provides the episode data and first annotation to a second human reviewer (1104), which either agrees with the first annotation or provides a revised annotation.

For episodes in category B, monitoring system 450 provides the episode data to a first human reviewer, who may be the same as or different from the first human reviewer that receives episode data for episodes in category A, and to a first machine reviewer 454 (1106). The first machine reviewer 454 may apply one or more machine learning models 452 to at least some of the episode data and, based on the output of the one or more machine learning models 452, determine a first annotation including whether one or more arrhythmias are detected in the episode data. Monitoring system 450 may provide the episode data to the first human reviewer and the first machine reviewer 454 in parallel, or may first provide the episode data to the first machine reviewer 454 and allow the first human reviewer to review the annotation made by the first machine reviewer 454. In some examples, monitoring system 450 may first provide the episode date to the first human reviewer, and provide the annotation from the first human reviewer as input to the first machine reviewer 454 for its analysis of the episode.

In any of these cases, monitoring system 450 determines whether the first annotations both indicate the same one or more arrhythmias (1108). Based on the first annotations agreeing (YES of 1108), monitoring system 450 may bypass the second reviewer. Based on at least some disagreement between the machine reviewer 454 and the first human reviewer (NO of 1108), monitoring system 450 may present the episode data and the first annotation to a second human reviewer (1104), which either agrees with the first annotations or provides a revised annotation.

The set of all arrhythmias that monitoring system 450 tries to identify in each episode can depend on: (i) trigger-reason, e.g., patient or auto-triggered and, for auto-triggered, which type of arrhythmia the medical device detected; and/or (ii) physician criteria that may be provided during programming of IMD 10 or via computing system 24, e.g., bradycardia of 4/4 beats <=40 beats-per-minute; or (iii) patient demographics. Examples of use of patient demographics includes setting a tachycardia and/or bradycardia threshold per patient age and maximal activity level, or tachycardia and bradycardia thresholds per heart failure status.

In some examples, auto-triggered episodes are processed by the machine reviewer 454 using one or more machine learning models 452 configured to detect various types of arrhythmia per annotation requirements specified by monitoring system 450 and/or one or more users. In some examples, the first human reviewer is blinded to annotation of machine reviewer 454, and if monitoring system 450 determines that both the first human and machine reviewers have flagged the same arrhythmias, the monitoring system may finalize the episode without subsequent review. In some examples, episodes where the machine reviewer 454 detect no arrhythmias may be presented in a multi-episode review screen for expedited review, e.g., as illustrated in FIGS. 9 and 10.

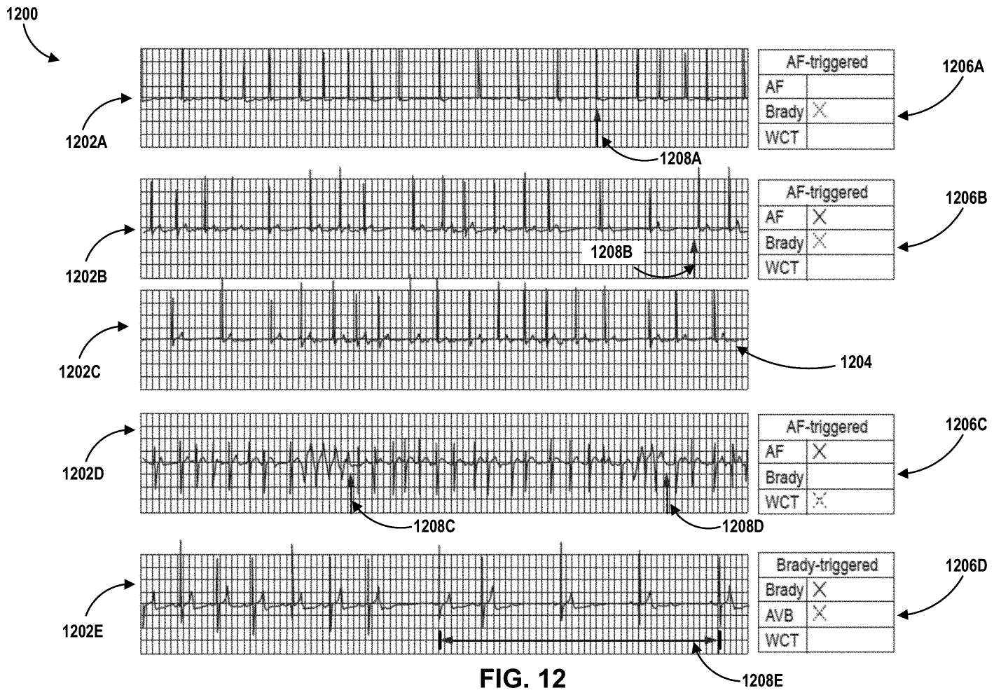

FIG. 12 is a diagram illustrating another example multi-episode review screen 1200 that may be displayed to a reviewer during an episode review workflow in accordance with the techniques of this disclosure. In the illustrated example, review screen 1200 includes episode data for episodes 1202A-1202E (collectively “episodes 1202”), including the cardiac EGMs 1204 for these episodes. In addition to the episode data, screen 1000 may include other information, such as annotations, e.g., by machine reviewer 454, that may aid in efficient review by the first human reviewer. The annotations may include features of the episode data identified by, and/or information derived from the episode data by machine reviewer 454, e.g., by one or more machine learning models 452. The information may be specific to the arrhythmia type that the medical devices and/or machine reviewer 454 detected or is intended to detect.

In the illustrated example, monitoring system 450 includes tables 1206A-1206D (collectively “tables 1206”) for episodes 1202A, 1202B, 1202D, and 1202E, respectively, on screen 1200. Each of tables 1206 includes a respective list of arrhythmia types for annotation for the given episode per the selection of monitoring system 450 and/or one or more users, as well as an indication of which of the arrhythmia types were detected by machine reviewer 454. Monitoring system 450 also includes markers 1208A-1208E (collectively “markers 1208”) on the cardiac EGMs for episodes 1202A, 1202B, 1202D, and 1202E indicating times during the episodes that machine reviewer 454 determined arrhythmias of the indicated types to have occurred.

For example, tables 1206A and 1206B for episodes 1202A and 1202B indicate that a machine reviewer 454 determined that a bradycardia occurred, and markers 1208A and 1208B indicate, in conjunction with the respective cardiac EGMs, the time of occurrence of the bradycardias within the episodes as determined by machine reviewer 454 using one or more machine learning models 452. Both episodes 1202A and 1202B were auto-triggered based on the medical device detecting atrial fibrillation. However, machine reviewer 454 did not detect atrial fibrillation in episode 1202A as indicated by table 1206A.

Table 1206C indicates that machine reviewer 454 detected wide complex tachycardia in addition to atrial fibrillation, which was the reason the medical device stored the episode. Markers 1208C and 1208D indicate locations within the episode at which machine reviewer 454 determined the wide complex tachycardias to have occurred. Similarly, table 1206D indicates that machine reviewer 454 detected atrioventricular block in addition to bradycardia, which was the reason the medical device stored the episode. Marker 1208E indicate a location within the episode at which machine reviewer 454 determined the atrioventricular block to have occurred.

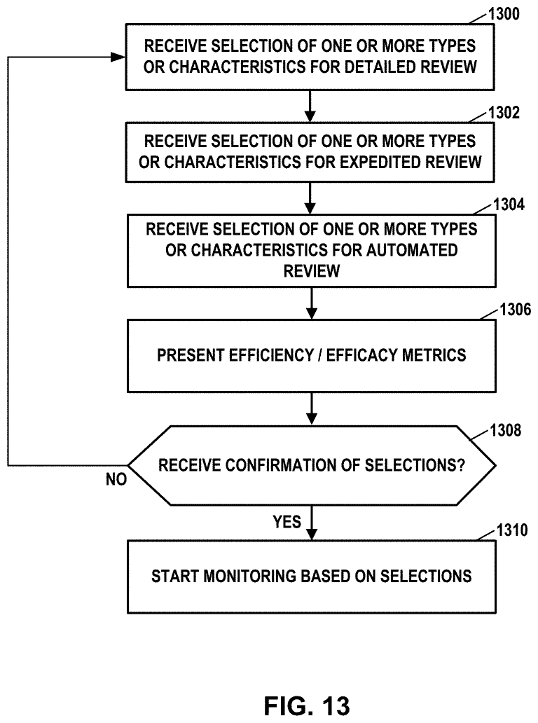

FIG. 13 is a flow diagram illustrating an example operation for configuring episode review workflows in accordance with the techniques of the disclosure. More particularly, FIG. 13 illustrates a technique whereby a clinician or other user may establish episode categories and specify aspects of the review workflows associated with those categories, e.g., for a particular clinic or practice group.

According to the example of FIG. 13, monitoring system 450 may receive user input selecting a first one or more episode types or characteristics for inclusion in a first category associated with a manual review workflow including episode annotation by a human reviewer (1300). Monitoring system 450 may receive user input selecting a second one or more episode types or characteristics for inclusion in a second category associated with an expedited review workflow comprising episode annotation by a machine reviewer comprising one or more machine learning models and the human reviewer (1302). Examples of manual and expedited review workflows are described with respect to FIGS. 6-8 and 11. Monitoring system 450 may receive user input selecting a third one or more episode types or characteristics for inclusion in a third category associated with an automated review workflow comprising episode annotation by the machine reviewer, e.g., to the exclusion of human reviewers (1304). Monitoring system 450 may present any of a variety of types of graphical user interface and instructions to prompt and receive such user input, e.g., via drop down menus, lists within associated radio buttons, or the like.

Based on the user's selections, monitoring system 450 may determine values of metrics of the efficiency and efficacy of the overall episode review processing, and present these values to the user (1306). Monitoring system 450 determine whether the user has confirmed the selections after reviewing the metric values (1308). If the user does not confirm the selections (NO of 1308), monitoring system 450 may present a graphical user interface for user modification of the selections (1300-1304). If the user confirms the selections (YES of 1308), monitoring system 450 may start review of episodes retrieved from medical devices, including IMD 10, based the categories and workflows established by the selections (1310).

For example, monitoring system 450 may determine at least one episode characteristic of an episode based on its episode data, and categorize the episode into one of the three categories based on the characteristic. The episode characteristics may comprise one or more of patient demographics, patient diagnoses, arrhythmia types, or whether the episode was patient-triggered or auto-triggered. In some examples, monitoring system 450 may prompt the user to modify the associations of the episode characteristics and the categories based on at least one of a schedule or a change in a condition of one or more patients, e.g., after a medication change or procedure. In some examples, a user may establish the associations of the episode characteristics and the categories on a per-patient or per-diagnoses basis.

In some examples, the category associated with manual episode review workflows may include any episodes that the user would like to review in detail, such as those episode types/patients where the user has additional monitoring follow-up needs (e.g., patient call-up during patient triggers) or prefer a detailed review (e.g., not miss any potential atrial fibrillation episode in cryptogenic stroke patients). There may be a tradeoff of efficiency gain for likelihood of misclassifications (false positives and false negatives) when automating the review of episodes with a machine reviewer 454 using machine learning models 452. Based on the use-case and algorithm performance, users can choose to automate the review of certain types of episodes, such as when monitoring patients for atrial fibrillation management where there is a large volume of atrial fibrillation-triggered episodes, and a machine learning model 452 having few false positive detections is viable. The efficiency and efficacy metrics may include estimates of total expected efficiency gain versus potential missed classifications based on historical data from other monitoring centers/clinics. Additionally, expedited review workflows, e.g., as described herein with respect to FIGS. 5-12, may provide efficiency gains while ensuring that all episodes are reviewed by at least one human technician/expert.

In some examples, if the user does not choose to establish associations between episode review workflows and episode categories as described with respect to FIG. 13, monitoring system 450 may utilize default categorizations for certain types of review workflows. For example, monitoring system 450 may default to manual review for some episode types, e.g., patient triggers, and expedited review for other episode types, e.g., auto-triggers. In some examples, monitoring system 450 may not use the completely automated workflow as a default for any episode category to retain some level of human oversight of episode review.

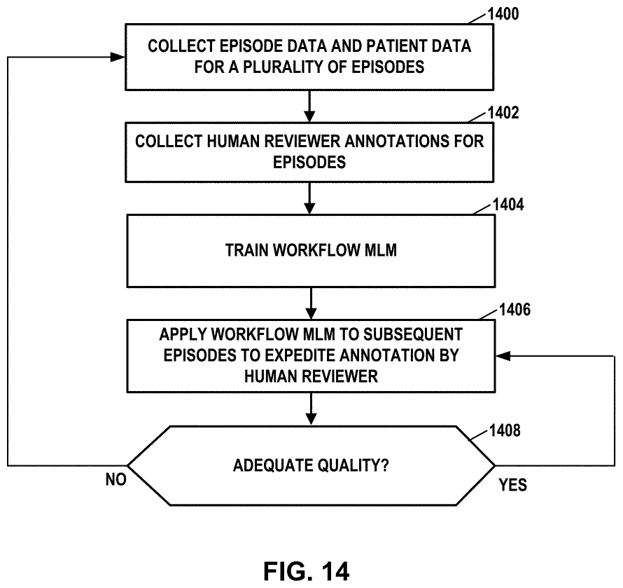

FIG. 14 is a flow diagram illustrating an example operation for utilizing a machine learning model to configure episode review workflows in accordance with the techniques of this disclosure. More particularly, FIG. 14 illustrates a technique whereby a machine learning model, e.g., neural network, may be trained to determine one or more workflows tailored for a particular clinic or practice group.

According to the example of FIG. 14, monitoring system 450 receives episode data for a plurality of episodes stored by a plurality of medical devices of a plurality of patients, wherein the episode data for each of the episodes comprises a respective cardiac electrogram (1400). For each of the episodes, monitoring system 450 may determine one or more patient characteristics or episode characteristics, and receive one or more annotations of the episode from a user (1402).

Monitoring system 450 trains one or more workflow machine learning models using the episode data, determined characteristics, and received annotations (1404), and applies the trained machine learning model to subsequent episodes to expedite annotation, e.g., by automatically establishing categories and workflows tailored to the clinic or practice group (1406). For example, monitoring system 450 may receive episode data for at least one subsequent episode stored by at least one of the plurality of medical devices, determine one or more patient characteristics or episode characteristics of the subsequent episode based on the episode data, and determine, by applying the machine learning model to the determined characteristics, a review workflow for the episode. Monitoring system 450 may present the episode data to one or more reviewers for annotation based on the determined workflow. Monitoring system 450 may assess, e.g., periodically, whether the trained workflow provides adequate review quality (1408), and continue applying (YES of 1408) or retrain (NO of 1408) the workflow machine learning model based on the assessment.

Examples according to FIG. 14 may include deep-learning of arrhythmia review workflows, as opposed to the techniques of FIGS. 5-13 for expediting or automating parts of established workflows. Patient characteristics may include patient demographics, such as age or gender, patient indications for use of the medical device or other patient diagnosis, comorbidities, or events, such as hospitalizations. Episode characteristics include the trigger reason, features of the cardiac EGM, activity level during the episode, and outputs from an intermediate layer of machine learning model 452 that determines whether one or more arrhythmias are present, the outputs from the intermediate layer indicating the relative probabilities of different arrhythmias over time, also referred to as a heatmap or a class activation map including class activation data. Monitoring system 450 may iterate the review workflow model during the learning phase for adequate quality, defined as workflow automation efficiency with minimal misclassifications. In addition to periodic assessments, quality may be reassessed in response to user input or input data type changes.

In some examples, the techniques of the disclosure include a system that comprises means to perform any method described herein. In some examples, the techniques of the disclosure include a computer-readable medium comprising instructions that cause processing circuitry to perform any method described herein.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques described herein.

### Example 1

A method comprising: receiving, by a computing system comprising processing circuitry and a storage medium, episode data for an episode stored by a medical device of a patient, wherein the episode data comprises a cardiac electrogram; categorizing, by the computing system, the episode into one category of a plurality of categories based on the episode data, the plurality of categories comprising at least a first category and a second category; selecting, by the computing system, one review workflow from a plurality of review workflows based on the category, each of the plurality of categories associated with a respective one of the plurality of review workflows; selecting, by the computing system, at least one first reviewer for the episode based on the selected review workflow; providing, by the computing system, the episode data to the at least one first reviewer; receiving, by the computing system, at least one first annotation of the episode by the at least one first reviewer, the at least one first annotation based on the provided episode data; determining, by the computing system, whether to provide the episode data to a second reviewer based on the selected review workflow; determining, by the computing system, whether to include the episode in an arrhythmia episode report based on the selected review workflow and the at least one first annotation; and outputting, by the computing system, the arrhythmia episode report to a user.

### Example 2

The method of example 1, wherein the episode data indicates whether the medical device stored the episode in response to user input or the medical device determining that the episode was one of a plurality of arrhythmia types, and wherein categorizing the episode comprises: categorizing the episode into the first category of the plurality of categories based on the episode data indicating that the medical device stored the episode in response to user input; and categorizing the episode into the second category of the plurality of categories based on the episode data indicating that the medical device stored the episode in response the medical device detecting one of the plurality of arrhythmia types.

### Example 3

The method of example 1, wherein the episode data indicates whether the medical device stored the episode in response to user input or the medical device determining that the episode was one of a plurality of arrhythmia types, and wherein categorizing the episode comprises: categorizing the episode into the first category of the plurality of categories based on the episode data indicating that the medical device stored the episode in response to user input or determining that the episode was one of a first subset of the plurality of arrhythmia types; and categorizing the episode into the second category of the plurality of categories based on the episode data indicating that the medical device stored the episode in response the medical device determining that the episode was one of a second subset of the plurality of arrhythmia types.

### Example 4

The method of example 3, further comprising, for each of the plurality of arrhythmia types, including the arrhythmia type in either the first subset or the second subset based on at least one of a prevalence of the arrhythmia type in the patient, a prevalence of false detections of the arrhythmia type by the medical device, or user input.

### Example 5

The method of any of examples 1 to 4, wherein categorizing the episode comprises categorizing the episode into the first category, wherein selecting the review workflow comprises selecting the review workflow associated with the first category from the plurality of review workflows, wherein selecting the at least one first reviewer comprises selecting a first human reviewer based on the review workflow associated with the first category, the method further comprising: providing the episode data to a second human reviewer based on the selected review workflow; and receiving, by the computing system, a second annotation of the episode by the second reviewer, the second annotation based on the provided episode data, and wherein determining whether to include the episode in the arrhythmia episode report comprises determining whether to include the episode in the arrhythmia episode report based on the selected review workflow and the at least one first annotation and the second annotation.

### Example 6

The method of any of examples 1 to 5, wherein categorizing the episode comprises categorizing the episode into the second category, wherein selecting the review workflow comprises selecting the review workflow associated with the second category from the plurality of review workflows, and wherein determining whether to provide the episode data to the second reviewer comprises determining whether to provide the episode data to the second reviewer based on the selected review workflow and the at least one first annotation.

### Example 7

The method of example 6, wherein determining whether to provide the episode data to the second reviewer comprises providing the episode data to the second reviewer based on the at least one first annotation indicating that the episode includes an arrhythmia.

### Example 8

The method of example 6 or 7, wherein determining whether to provide the episode data to the second reviewer comprises bypassing the second reviewer based on the at least one first annotation indicating “no arrhythmia” for the episode.

### Example 9

The method of any of examples 6 to 8, wherein selecting the at least one first reviewer comprises selecting a first human reviewer and a first machine reviewer comprising one or more machine learning models, wherein providing the episode data to the at least one first reviewer comprises applying at least some of the episode data to the one or more machine learning models, the at least some of the episode data including the cardiac electrogram, wherein receiving the at least one first annotation comprises receiving, from the first machine reviewer, a machine annotation based on output of the one or more machine learning models in response to the application of the at least some of the episode data to the one or more machine learning models, and wherein the at least one first annotation comprises the machine annotation and an annotation from the first human reviewer.

### Example 10

The method of example 9, further comprising determining, based on the selected review workflow, whether to provide the machine annotation to the first human reviewer prior to receiving the annotation from the first human reviewer.

### Example 11

The method of example 9 or 10, wherein determining whether to provide the episode data to the second reviewer comprises bypassing the second reviewer based on agreement between the machine annotation and the annotation from the first human reviewer.

### Example 12

The method of example 11, wherein bypassing the second reviewer comprises bypassing the second reviewer based on both the machine annotation and the annotation from the first reviewer indicating “no arrhythmia” for the episode.

### Example 13

The method of example 11, wherein bypassing the second reviewer comprises bypassing the second reviewer based on both the machine annotation and the annotation from the first reviewer indicating a same one or more arrhythmias in the episode.

### Example 14

The method of example 9 or 10, wherein determining whether to provide the episode data to the second reviewer comprises providing the episode data to the second reviewer based on disagreement between the machine annotation and the annotation from the first human reviewer.

### Example 15

The method of example 14, wherein providing the episode data to the second reviewer comprises providing the episode data to the second reviewer based on only one of the machine annotation and the annotation from the first reviewer indicating “no arrhythmia” for the episode.

### Example 16

The method of example 14, wherein providing the episode data to the second reviewer comprises providing the episode data to the second reviewer based on only the machine annotation indicating “no arrhythmia” for the episode.

### Example 17

The method of example 11, wherein providing the episode data to the second reviewer comprises providing the episode data to the second reviewer based the machine annotation and the annotation from the first reviewer indicating a different one or more arrhythmias in the episode.

### Example 18

The method of any of claims 12, 15 and 16, wherein the one or more machine learning models comprise a machine learning model configured to classify the episode as being one of an arrhythmia classification or a no arrhythmia classification based on the at least some of the episode data.

### Example 19

The method of example 18, wherein categorizing the episode into the second category comprises categorizing the episode into the second category based on the episode data indicating that the medical device stored the episode in response the medical device determining that the episode included an arrhythmia of an arrhythmia type, the method further comprising: determining a prevalence of false detection of arrhythmias of the arrhythmia type by the medical device; determining a positive predictive value of no arrhythmia for a plurality of machine learning models; comparing the prevalence of false detection to the positive predictive values; and selecting the machine learning model based on the comparison.

### Example 20

The method of example 13 or 17, wherein the one or more machine learning models comprise one or more machine learning models configured to identify one or more arrhythmias of one or more arrhythmia types in the episode based on the at least some of the episode data.

### Example 21

The method of any of examples 9 to 20, wherein providing the episode data to the at least one first reviewer comprises simultaneously presenting episode data and machine annotations for a plurality of episodes to the first reviewer.

### Example 22

The method of example 21, wherein the machine annotations for each of the plurality of episodes include an indication of no arrhythmia, and simultaneously presenting the episode data and machine annotations for the plurality of episodes comprises simultaneously presenting the episode data and machine annotations for the plurality of episodes based on the indications of no arrhythmia.

### Example 23

A method comprising associating, by a computing system comprising processing circuitry and a storage medium, based on user input, a respective one or more episode characteristics with each of a plurality of episode categories, wherein the plurality of episode categories comprises: a first category associated with a manual review workflow comprising episode annotation by a human reviewer; a second category associated with an automated review workflow comprising episode annotation by a machine reviewer comprising one or more machine learning models; and a third category associated with an expedited review workflow comprising annotation by the machine reviewer and the human reviewer. The method further comprises: receiving, by the computing system, episode data for an episode stored by a medical device of a patient, wherein the episode data comprises a cardiac electrogram; determining, by the computing system, at least one episode characteristic of episode based on the episode data; categorizing, by the computing system, the episode into one of the first category, the second category, or the third category based on the at least one episode characteristic; providing, by the computing system, the episode data to at least one of the human reviewer or the machine reviewer based on the workflow associated with the category into which the episode was categorized; receiving, by the computing system, at least one annotation of the episode by the at least one of the human reviewer or the machine reviewer, the at least one first annotation based on the provided episode data; determining, by the computing system, whether to include the episode in an arrhythmia episode report based on the selected review workflow and the at least one annotation; and outputting, by the computing system, the arrhythmia episode report to a user.

### Example 24

The method of example 23, wherein the episode characteristics comprise one or more of patient demographics, patient diagnoses, arrhythmia types, or whether the episode data was stored by a medical device in response to user input or detection of an arrhythmia.

### Example 25

The method of example 23 or 24, further comprising prompting, by the computing system, the user to modify the associations of the episode characteristics and the categories based on at least one of a schedule or a change in a condition of one or more patients.

### Example 26

The method of any of examples 23 to 25, further comprising: determining, by the computing system, at least one of review efficiency metrics or review efficacy metrics based on the associations; and presenting, by the computing system, the at least one of the review efficiency metrics or review efficacy metrics to the user.

### Example 27

The method of example 26, further comprising, by the computing system, and after presenting the at least one of the review efficiency metrics or review efficacy metrics to the user, prompting the user to either accept or modify the associations of the episode characteristics and the categories.

### Example 28

The method of any of examples 23 to 27, further comprising selecting, by the computing system, one or more patients from among a plurality of patients based on user input, wherein associating the respective one or more episode characteristics with each of the plurality of episode categories comprises associating the respective one or more episode characteristics with each of the plurality of episode categories for the selected patients.

### Example 29

A method comprising: receiving, by a computing system comprising processing circuitry and a storage medium, episode data for a plurality of episodes stored by a plurality of medical devices of a plurality of patients, wherein the episode data for each of the episodes comprises a respective cardiac electrogram; for each of the episodes, determining, by the computing system, one or more patient characteristics or episode characteristics based on the episode data; for each of the episodes, receiving, by the computing system, one or more annotations from a user; training, by the computing system, one or more machine learning models using the episode data, determined characteristics, and received annotations; receiving, by the computing system, episode data for at least one subsequent episode stored by at least one of the plurality of medical devices; determining, by the computing system, one or more patient characteristics or episode characteristics of the subsequent episode based on the episode data; determining, by the computing system applying the machine learning model to the determined characteristics, a review workflow for the episode; and presenting, by the computing device, the episode data to one or more reviewers for annotation based on the determined workflow.

Various examples have been described. These and other examples are within the scope of the following claims.

What is claimed is:

1. A method comprising:
receiving, by a computing system comprising processing circuitry and a storage medium, episode data for an episode stored by a medical device of a patient, wherein the episode data comprises a cardiac electrogram;
categorizing, by the computing system, the episode into one category of a plurality of categories based on the episode data, the plurality of categories comprising at least a first category and a second category;
selecting, by the computing system, one review workflow from a plurality of review workflows based on the category, each of the plurality of categories associated with a respective one of the plurality of review workflows;
selecting, by the computing system, at least one first reviewer for the episode based on the selected review workflow;
providing, by the computing system, the episode data to the at least one first reviewer;
receiving, by the computing system, at least one first annotation of the episode by the at least one first reviewer, the at least one first annotation based on the provided episode data;
determining, by the computing system, whether to provide the episode data to a second reviewer based on the selected review workflow;
determining, by the computing system, whether to include the episode in an arrhythmia episode report based on the selected review workflow and the at least one first annotation; and
outputting, by the computing system, the arrhythmia episode report to a user.

2. The method of claim 1, wherein the episode data indicates whether the medical device stored the episode in response to user input or the medical device determining that the episode was one of a plurality of arrhythmia types, and wherein categorizing the episode comprises one of:
categorizing the episode into the first category of the plurality of categories based on the episode data indicating that the medical device stored the episode in response to user input; or
categorizing the episode into the second category of the plurality of categories based on the episode data indicating that the medical device stored the episode in response the medical device detecting one of the plurality of arrhythmia types.

3. The method of claim 1, wherein the episode data indicates whether the medical device stored the episode in response to user input or the medical device determining that the episode was one of a plurality of arrhythmia types, and wherein categorizing the episode comprises one of:
categorizing the episode into the first category of the plurality of categories based on the episode data indicating that the medical device stored the episode in response to user input or determining that the episode was one of a first subset of the plurality of arrhythmia types; or
categorizing the episode into the second category of the plurality of categories based on the episode data indicating that the medical device stored the episode in response the medical device determining that the episode was one of a second subset of the plurality of arrhythmia types.

4. The method of claim 1,
wherein categorizing the episode comprises categorizing the episode into the first category,
wherein selecting the review workflow comprises selecting the review workflow associated with the first category from the plurality of review workflows,
wherein selecting the at least one first reviewer comprises selecting a first human reviewer based on the review workflow associated with the first category,
the method further comprising:
providing the episode data to a second human reviewer based on the selected review workflow; and
receiving, by the computing system, a second annotation of the episode by the second reviewer, the second annotation based on the provided episode data, and
wherein determining whether to include the episode in the arrhythmia episode report comprises determining whether to include the episode in the arrhythmia episode report based on the selected review workflow and the at least one first annotation and the second annotation.

5. The method of claim 1,
wherein categorizing the episode comprises categorizing the episode into the second category,
wherein selecting the review workflow comprises selecting the review workflow associated with the second category from the plurality of review workflows, and
wherein determining whether to provide the episode data to the second reviewer comprises determining whether to provide the episode data to the second reviewer based on the selected review workflow and the at least one first annotation.

6. The method of claim 5, wherein determining whether to provide the episode data to the second reviewer comprises one of:
providing the episode data to the second reviewer based on the at least one first annotation indicating that the episode includes an arrhythmia; or
bypassing the second reviewer based on the at least one first annotation indicating “no arrhythmia” for the episode.

7. The method of claim 5,
wherein selecting the at least one first reviewer comprises selecting a first human reviewer and a first machine reviewer comprising one or more machine learning models,
wherein providing the episode data to the at least one first reviewer comprises applying at least some of the episode data to the one or more machine learning models, the at least some of the episode data including the cardiac electrogram,
wherein receiving the at least one first annotation comprises receiving, from the first machine reviewer, a machine annotation based on output of the one or more machine learning models in response to the application of the at least some of the episode data to the one or more machine learning models, and
wherein the at least one first annotation comprises the machine annotation and an annotation from the first human reviewer.

8. The method of claim 7, wherein determining whether to provide the episode data to the second reviewer comprises one of:
bypassing the second reviewer based on agreement between the machine annotation and the annotation from the first human reviewer; or
providing the episode data to the second reviewer based on disagreement between the machine annotation and the annotation from the first human reviewer.

9. The method of claim 8, wherein bypassing the second reviewer comprises bypassing the second reviewer based on both the machine annotation and the annotation from the first reviewer indicating the same one or more arrhythmias in the episode or “no arrhythmia” for the episode.

10. The method of claim 9, wherein the one or more machine learning models comprise a machine learning model configured to classify the episode as being one of an arrhythmia classification or a no arrhythmia classification based on the at least some of the episode data.

11. The method of claim 10, wherein categorizing the episode into the second category comprises categorizing the episode into the second category based on the episode data indicating that the medical device stored the episode in response the medical device determining that the episode included an arrhythmia of an arrhythmia type, the method further comprising:
determining a prevalence of false detection of arrhythmias of the arrhythmia type by the medical device;
determining a positive predictive value of no arrhythmia for a plurality of machine learning models;
comparing the prevalence of false detection to the positive predictive values; and
selecting the machine learning model based on the comparison.

12. The method of claim 1, further comprising associating, by the computing system, each of the plurality of categories with a respective one or more episode characteristics, wherein categorizing the episode into one category of the plurality of categories comprises:
determining, by the computing system, at least one episode characteristic of episode based on the episode data; and
categorizing, by the computing system, the episode into the category based on the at least one episode characteristic.

13. The method of claim 12, wherein the episode characteristics comprise one or more of patient demographics, patient diagnoses, arrhythmia types, or whether the episode data was stored by a medical device in response to user input or detection of an arrhythmia.

14. The method of claim 12, further comprising:
determining, by the computing system, at least one of review efficiency metrics or review efficacy metrics based on the associations;
presenting, by the computing system, the at least one of the review efficiency metrics or review efficacy metrics to the user; and
prompting, by the computing device, and after presenting the at least one of the review efficiency metrics or review efficacy metrics to the user, the user to either accept or modify the associations of the episode characteristics and the categories.

15. A computing system comprising processing circuitry and a storage medium, wherein the processing circuitry is configured to:
receive episode data for an episode stored by a medical device of a patient, wherein the episode data comprises a cardiac electrogram;
categorize the episode into one category of a plurality of categories based on the episode data, the plurality of categories comprising at least a first category and a second category;
select one review workflow from a plurality of review workflows based on the category, each of the plurality of categories associated with a respective one of the plurality of review workflows;
select at least one first reviewer for the episode based on the selected review workflow;
provide the episode data to the at least one first reviewer;
receive at least one first annotation of the episode by the at least one first reviewer, the at least one first annotation based on the provided episode data;
determine whether to provide the episode data to a second reviewer based on the selected review workflow;
determine whether to include the episode in an arrhythmia episode report based on the selected review workflow and the at least one first annotation; and
output the arrhythmia episode report to a user.

16. The computing system of claim 15, wherein the episode data indicates whether the medical device stored the episode in response to user input or the medical device determining that the episode was one of a plurality of arrhythmia types, and wherein the processing circuitry is configured to:
categorize the episode into the first category of the plurality of categories based on the episode data indicating that the medical device stored the episode in response to user input; and
categorize the episode into the second category of the plurality of categories based on the episode data indicating that the medical device stored the episode in response the medical device detecting one of the plurality of arrhythmia types.

17. The computing system of claim 15, wherein the episode data indicates whether the medical device stored the episode in response to user input or the medical device determining that the episode was one of a plurality of arrhythmia types, and wherein the processing circuitry is configured to:
categorize the episode into the first category of the plurality of categories based on the episode data indicating that the medical device stored the episode in response to user input or determining that the episode was one of a first subset of the plurality of arrhythmia types; and
categorize the episode into the second category of the plurality of categories based on the episode data indicating that the medical device stored the episode in response the medical device determining that the episode was one of a second subset of the plurality of arrhythmia types.

18. The computing system of claim 15, wherein when the processing circuitry categorizes the episode into the first category, selects the review workflow associated with the first category from the plurality of review workflows, and selects a first human reviewer based on the review workflow associated with the first category, the processing circuitry is configured to:
provide the episode data to a second human reviewer based on the selected review workflow;
receive a second annotation of the episode by the second reviewer, the second annotation based on the provided episode data; and
determine whether to include the episode in the arrhythmia episode report based on the selected review workflow and the at least one first annotation and the second annotation.

19. The computing system of claim 15, wherein when the processing circuitry categorizes the episode into the second category, and selects the review workflow associated with the second category from the plurality of review workflows, the processing circuitry is configured to determine whether to provide the episode data to the second reviewer based on the selected review workflow and the at least one first annotation.

20. The computing system of claim 19, wherein the processing circuitry is configured to:
provide the episode data to the second reviewer based on the at least one first annotation indicating that the episode includes an arrhythmia; and
bypass the second reviewer based on the at least one first annotation indicating “no arrhythmia” for the episode.

21. The computing system of claim 19,
wherein, to select the at least one first reviewer, the processing circuitry is configured to select a first human reviewer and a first machine reviewer comprising one or more machine learning models,
wherein, to provide the episode data to the at least one first reviewer, the processing circuitry is configured to apply at least some of the episode data to the one or more machine learning models, the at least some of the episode data including the cardiac electrogram,
wherein, to receive the at least one first annotation, the processing circuitry is configured to receive, from the first machine reviewer, a machine annotation based on output of the one or more machine learning models in response to the application of the at least some of the episode data to the one or more machine learning models, and
wherein the at least one first annotation comprises the machine annotation and an annotation from the first human reviewer.

22. The computing system of claim 21, wherein the processing circuitry is configured to:
bypass the second reviewer based on agreement between the machine annotation and the annotation from the first human reviewer; and
provide the episode data to the second reviewer based on disagreement between the machine annotation and the annotation from the first human reviewer.

23. The computing system of claim 22, wherein the processing circuitry is configured to bypass the second reviewer based on both the machine annotation and the annotation from the first reviewer indicating the same one or more arrhythmias in the episode or “no arrhythmia” for the episode.

24. The computing system of claim 23, wherein the one or more machine learning models comprise a machine learning model configured to classify the episode as being one of an arrhythmia classification or a no arrhythmia classification based on the at least some of the episode data.

25. The computing system of claim 24, wherein the processing circuitry is configured to:
categorize the episode into the second category based on the episode data indicating that the medical device stored the episode in response the medical device determining that the episode included an arrhythmia of an arrhythmia type;
determine a prevalence of false detection of arrhythmias of the arrhythmia type by the medical device;
determine a positive predictive value of no arrhythmia for a plurality of machine learning models;
compare the prevalence of false detection to the positive predictive values; and
select the machine learning model based on the comparison.

26. The computing system of claim 15, wherein the processing circuitry is configured to associate each of the plurality of categories with a respective one or more episode characteristics, and categorize the episode into one category of the plurality of categories by at least:
determining at least one episode characteristic of episode based on the episode data; and
categorize the episode into the category based on the at least one episode characteristic.

27. The computing system of claim 26, wherein the episode characteristics comprise one or more of patient demographics, patient diagnoses, arrhythmia types, or whether the episode data was stored by a medical device in response to user input or detection of an arrhythmia.

28. The computing system of claim 26, wherein the processing circuitry is configured to:
determine at least one of review efficiency metrics or review efficacy metrics based on the associations;
present the at least one of the review efficiency metrics or review efficacy metrics to the user; and
prompt, after presenting the at least one of the review efficiency metrics or review efficacy metrics to the user, the user to either accept or modify the associations of the episode characteristics and the categories.

29. A non-transitory computer-readable medium comprising instructions, that when executed by processing circuitry of a computing system, cause the computing system to:
receive episode data for an episode stored by a medical device of a patient, wherein the episode data comprises a cardiac electrogram;
categorize the episode into one category of a plurality of categories based on the episode data, the plurality of categories comprising at least a first category and a second category;
select one review workflow from a plurality of review workflows based on the category, each of the plurality of categories associated with a respective one of the plurality of review workflows;
select at least one first reviewer for the episode based on the selected review workflow;
provide the episode data to the at least one first reviewer;
receive at least one first annotation of the episode by the at least one first reviewer, the at least one first annotation based on the provided episode data;
determine whether to provide the episode data to a second reviewer based on the selected review workflow;
determine whether to include the episode in an arrhythmia episode report based on the selected review workflow and the at least one first annotation; and
output the arrhythmia episode report to a user.
